(19) United States
(12) Patent Application Publication
(10) Pub. No.: US 2015/0282695 A1
(43) Pub. Date: Oct. 8, 2015

(54) ENDOSCOPES

(71) Applicant: AGENCY FOR SCIENCE, TECHNOLOGY AND RESEARCH, Connexis, Singapore (SG)

(72) Inventors: Hsien Ts'ung Tay, Singapore (SG); Joon Yin Phoon, Singapore (SG); Zhao Ying Pearline Teo, Singapore (SG); Tze Kiat Ng, Singapore (SG)

(73) Assignee: Agency for Science, Technology and Research, Singapore (SG)

(21) Appl. No.: 14/443,455

(22) PCT Filed: Dec. 11, 2013
(86) PCT No.: PCT/SG2013/000528

§ 371 Date: May 18, 2015

(30) Foreign Application Priority Data
Dec. 11, 2012 (SG) 201209102-1

(51) Int. Cl. A61B 1/00; A61B 1/12

(52) U.S. Cl. CPC A61B 1/00133; A61B 1/126; A61B 1/00195; A61B 1/00142

(57) ABSTRACT

In various embodiments of the present disclosure, there is provided an endoscope including: an elongate shaft body; an optical scope provided within the elongate shaft body, the optical scope comprising an optical aperture provided at the distal end of the optical scope, corresponding to a distal end of the elongate shaft body; an aperture cover provided at the distal end of the elongate shaft body for shielding the optical aperture from debris external to the elongate shaft body; and an actuation system provided at a proximal end of the elongate shaft body; wherein the actuation system drives the aperture cover to rotate.

### CROSS-REFERENCE TO RELATED APPLICATIONS

The present application claims the benefit of the Singapore patent application No. 201209102-1, filed on 11 Dec. 2012, the entire contents of which are incorporated herein by reference for all purposes.

### TECHNICAL FIELD

The present disclosure describes embodiments generally relating to an endoscope, the endoscope including a system for improving visualization during operation of the endoscope.

### BACKGROUND

Minimally invasive surgery (MIS) is a field of medical procedures which typically involves the use of entering the body through the skin or a body cavity or anatomical opening, with the intention of reducing, as far as possible, damage to body structures. MIS generally encompasses laparoscopic, robotic, robotic-assisted laparoscopic, endoscopic, single-port and natural orifice surgery, and is becoming increasingly adopted as facilitative technologies progress, surgeon acceptance grows, and patient demand increases. MIS brings with it the advantages of reduced pain and faster postoperative recoveries, and in certain instances, improved cosmesis and even increased visualization or operative dexterity as compared with open surgery.

The setup in MIS typically involves a port through which an imaging device is inserted in the body to enable intracavity visualization. Such an imaging device which enters the body for visualization is considered an endoscope, and for which as a category, imaging devices crafted for more specific purposes, such as arthroscopes, laparoscopes, rectoscopes, amongst other tools fall under. Endoscopes typically include a rigid or flexible tube in extension of an imaging system, and it is noted that endoscopes now extend to uses which stretch beyond typical medical procedures, for example internal inspection of complex technical solution, or examination of explosive devices by bomb disposal personnel, etc.

Endoscopes as utilized in MIS usually include a combination light source for illumination and can be inserted in to the body through cavities into the abdomen, pelvis, or a subcutaneous space created by surgical separation of tissue planes. Endoscopes can also include an air insufflation port, and two or more additional working ports through which specialized surgical instruments can be inserted for manipulation, dissection and hemostasis.

MIS generally poses specific challenges as compared with open surgery. The working space is limited, and visualization is dependent on an optical imaging system or camera in the endoscope, which has a restricted field of view.

Further, multiple varieties of debris may impinge on the lens of the optical imaging system or lens of the scope, or the tip of the scope, preventing the surgeon from clearly viewing the surgical field. Such debris can include water vapor from a humid body cavity, vaporized tissue particles from dissection, blood, and smear from fat and viscera. The latter becomes more prevalent when operating in narrow cavities such as the pelvis, retro-peritoneum, thoracic cavity, nasal sinuses etc.

Water vapor fogging and vaporized tissue are probably the most common sources of lens fouling or soiling. However, blood from vessel injury can be considered the most dangerous as a scenario. When significant bleeding is encountered during surgery, reduction or cessation of blood flow is critical. However, for effective hemostasis to take place, it is imperative to have rapid access to the bleeding vessel and precise visualization of the bleeding point or points for accurate placement of the hemostatic tool. Therefore, if spurting blood impinges on the scope lens during such an attempt at hemostasis, precious time could be lost while attempting to restore visualization, with potentially dire consequences for the patient.

At present, when visualization is impaired, surgical workflow stops. The scope is removed from the patient, and the tip cleaned with gauze. Typically, the tip is then submerged in a thermos of warm water then dried again with gauze before re-insertion into the patient. The whole process takes about one minute, and may be repeated anywhere from two to ten times in an hour throughout a surgical procedure.

Laparoscope sheaths have been developed with fluid irrigation channels that enable lens cleaning without scope removal. However, this still requires a brief cessation in surgical workflow while the cleaning is taking place. Preventing the lens from being fouled allows surgeons the luxury of a constant, clear view. However, the gentle, air currents provided with the fluid irrigation channel sheath are not particularly effective against blood splatter, and can fail to protect the lens during the most critical moments of the surgery.

There is as such a desire for a system for the improving of visualization of endoscopes during operation.

### SUMMARY

According to various embodiments in the present disclosure, there is provided an endoscope including: an elongate shaft body; an optical scope provided within the elongate shaft body, the optical scope including an optical aperture provided at the distal end of the optical scope, corresponding to a distal end of the elongate shaft body; an aperture cover provided at the distal end of the elongate shaft body for shielding the optical aperture from debris external to the elongate shaft body; and an actuation system provided at a proximal end of the elongate shaft body; wherein the actuation system drives the aperture cover to rotate.

### BRIEF DESCRIPTION OF THE DRAWINGS

In the drawings, like reference characters generally refer to the same parts throughout the different views. The drawings are not necessarily to scale, with emphasis instead generally being placed upon illustrating the principles of the present disclosure. It is to be noted that the accompanying drawings illustrate only examples of embodiments of this disclosure and are therefore not to be considered limiting of its scope, for the disclosure may admit to other equally effective embodiments. In the following description, various embodiments of the disclosure are described with reference to the following drawings, in which:

FIG. 1A illustrates an endoscope according to an embodiment;

FIG. 1B illustrates an exemplary embodiment of an endoscope in demonstrating rotational speed calculation.

FIG. 2A illustrates a perspective view of a portion of the endoscope according to various embodiments;

FIG. 2B illustrates a cross-sectional portion of the distal end of the endoscope of FIG. 2A;

FIG. 2C illustrates a blown up cross-sectional view of the housing of FIG. 2A;

FIG. 2D illustrates a blown up perspective assembly view with the components of the housing of FIG. 2C laid out;

FIG. 3 illustrates a cross-sectional close-up view of a distal end of the endoscope of FIG. 2A;

FIG. 4 illustrates an endoscope with a fluid flow actuation system according to an embodiment;

FIG. 4B illustrates a cross-sectional view of the distal end of the endoscope of FIG. 4A;

FIG. 4C is a forward perspective view of the endoscope of FIG. 4A;

FIG. 5A illustrates a front view of an endoscope including a fluid flow feature for visualization improvement according to an embodiment;

FIG. 5B illustrates a cross-sectional view of the endoscope of FIG. 5A;

FIG. 6 illustrates a perspective view of an endoscope including a fluid column feature for visualization improvement according to an embodiment;

FIGS. 7A to 7D illustrate endoscopes including a fluid column feature and in varying degrees of angulation according to various embodiments;

FIGS. 8A to 8D illustrate an endoscope including a safety feature according to various embodiments;

FIG. 9A illustrates a front view of an endoscope including a wiper element feature for visualization improvement according to an embodiment;

FIG. 9B illustrates a cross-sectional view of the endoscope of FIG. 9A;

FIG. 10A illustrates a front view of the endoscope with the deployable wiper 1060 in an unengaged position;

FIG. 10B is a cross-sectional view of the endoscope with the deployable wiper in the unengaged position;

FIG. 10C illustrates a front view of the endoscope with the deployable wiper 1060 in an engaged position;

FIG. 10D is a cross-sectional view of the endoscope in the engaged position;

FIGS. 11A and 11B illustrate an endoscope including a moving wiper feature for visualization improvement according to an embodiment;

FIG. 12 illustrates a front view of an endoscope including a double wiper feature for visualization improvement according to an embodiment;

FIGS. 13A to 13C illustrate an endoscope including a displaceable wiper feature for visualization improvement according to an embodiment;

FIG. 14A illustrates a front view of an endoscope with a displaceable wiper in a first position;

FIG. 14B illustrates a front view of an endoscope with a displaceable wiper in transition from a first position to a second position;

FIG. 14C illustrates a front view of an endoscope with a displaceable wiper at a second position after transition from a first position;

FIGS. 15A-15E illustrate an endoscope including a spoolable wiper feature for visualization improvement according to an embodiment;

FIG. 16A illustrates a cross-sectional view of an endoscope including a next spoolable wiper feature for visualization improvement according to an embodiment;

FIG. 16B illustrates a spoolable wiper element according to an embodiment;

FIG. 17 illustrates an endoscope including a spoolable wiper feature including extendable arms for visualization improvement according to an embodiment;

FIGS. 18A-18C illustrate an endoscope including a spoolable wiper feature including a tapered sheath guide for visualization improvement according to an embodiment; and

FIG. 19 illustrates a block schematic of an endoscope according to an embodiment of the present disclosure.

### DETAILED DESCRIPTION

Embodiments of a method and system for communication channel distribution are described in detail below with reference to the accompanying figures. However, it should be understood that the disclosure is not limited to specific described embodiments. It will be appreciated that the embodiments described below can be modified in various aspects, features, and elements, without changing the essence of the disclosure. Further, any reference to various embodiments shall not be construed as a generalization of any inventive subject matter disclosed herein and shall not be considered to be an element or limitation of the appended claims except where explicitly recited in a claim(s).

According to various embodiments, depiction of a given element or consideration or use of a particular element number in a particular FIG. or a reference thereto in corresponding descriptive material can encompass the same, an equivalent, or an analogous element or element number, identified in another. FIG. or descriptive material associated therewith. The use of “I” herein means “and/or” unless specifically indicated otherwise.

The present disclosure can describe embodiments of a system or apparatus which can be operable in various orientations, and it thus should be understood that any of the terms “top”, “bottom”, “base”, “down”, “sideways”, “downwards” etc., when used in the following description are used for convenience and to aid understanding of relative positions or directions, and not intended to limit the orientation of a system or apparatus. It is also noted that the term “distal” is used to indicate a location or a portion situated away from a point of origin and the term “proximal” is used to indicate a location or a portion situated toward the point of origin.

According to various embodiments, an endoscope is provided with a capability to maintain visualization during minimally invasive surgery. According to various embodiments, the improved endoscope includes a rotating shield at the distal tip of the endoscope. Such a rotating shield maintains visualization by seeking to prevent impingement of debris on the optical lens or the optical aperture of the endoscope. Rotation of the shield dissipates large fluid droplets and remaining particles have a minimal effect on visual quality. In various embodiments, the endoscope includes additional elements or capabilities to augment the rotating shield. Such additional elements are useful, as centrifugal force, on its own, may be insufficient to handle large quantities of debris.

Further, according to the present disclosure, an improved endoscope is provided with a small sheath form factor. Such a form factor in the improved endoscope in various embodiments does not deviate greatly from the form factor of a comparable standard or typical endoscope, and allows the use of standard-sized ports, for example a standard-sized laparoscopic port of about 12-15 mm, for insertion into a body.

According to various embodiments, the improvements to the endoscope, for example, the rotatable aperture cover, can be provided in the form factor or housing of a standard-sized endoscope. In other embodiments, a sheath may be provided, which fits over the endoscope, to provide an improved endoscope according to the present disclosure. The endoscope according to various embodiments may provide an adaptation to angled scopes.

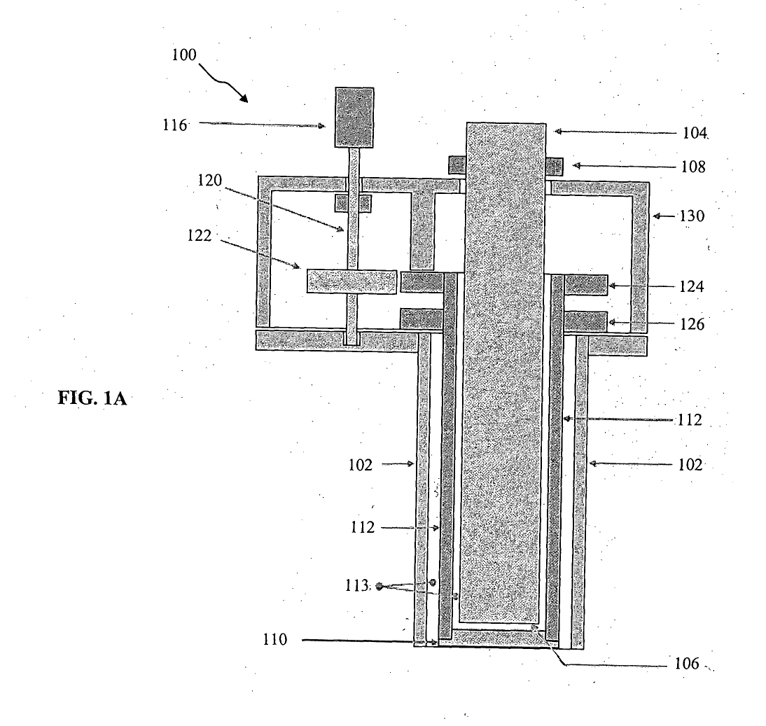

FIG. 1A illustrates an endoscope according to an embodiment. Endoscope 100 is provided with a capability to maintain visualization during minimally invasive surgery according to the present disclosure. The endoscope 100 includes an elongate shaft body or outer sheath 102 which houses the internal components of the endoscope 100. The outer sheath 102 is elongate to allow for a surgeon to operate for access and manipulation in a patient's body during MIS. In embodiments, the outer sheath 102 can be coupled to a manipulation apparatus which allows for an operating surgeon to manipulate a flexible endoscope in a patient's body. In embodiments, the outer sheath 102 is rigid and includes capability for directional manipulation of the distal end of the endoscope 100. According to various embodiments, the aperture cover and actuation system can be built-into the endoscope. In various embodiments, the aperture cover and actuation system can be separate modules that can be fitted onto the optical elements of an endoscope to form an endoscope system.

In embodiments, the outer sheath 102 houses an optical scope 104, which is arranged to be manipulated in accordance with the outer sheath 102 during operation. Scope 104 includes an optical imaging system or can be considered as part of an optical imaging system. In embodiments, the scope 104 typically includes a fiber optic bundle coupled to an image capture system at the proximal end of the scope 104. Distally, the fiber optic bundle is arranged to receive and transfer light and image details in an observation area through an optical aperture 106. In embodiments, the scope 104 could include a plurality of lenses which allow the conveyance of light and imaging from the distal end of the scope 104 to an optical imaging processing system.

In embodiments, the distal end of the scope 104 can include an optical lens which provides clarity and focus on the observation area. The distal end of the scope 104 can also include an imaging device, such as a charge-coupled device, which can allow for the additional image processing capabilities for the optical imaging system, including automatic focusing and light detection and adaptation, amongst others. Any optical apparatus provided over the optical aperture of the fiber optic bundle can also be considered to provide a further optical aperture of the scope 104.

According to various embodiments, the endoscope 100 includes a lens cover or aperture cover 110 provided at the distal end of the outer sheath 102. The lens cover 110 is provided over the lens or aperture of the scope 104 and provides a function of protecting the scope and maintaining visualization during operation. Such a lens cover 110 in performing an optical function is intended to be transparent and arranged as not to be an impediment to optical imaging. In embodiments, the lens cover 110 is an acrylic lens cover, but can be of any other construct. Acrylic is chosen because of its superior optical properties, but any other optically clear material such as polystyrene, polypropylene or PMMA could be utilized. According to various embodiments, the lens cover should be sized to substantially cover the area of the objective lens or aperture 106 of the scope 104. In embodiments, the lens cover 110 covers at least 50% of the objective area of the optical aperture 106. In an embodiment, the lens cover 110 is a clear acrylic disc, and which is 6.58 mm in diameter, the endoscope configured for use as a laparoscope. The circular shape of the disc assures the absence of sharp corners, which could potentially cause tissue injury should the rotation of the lens cover get caught in tissue.

According to various embodiments, the edges of the lens cover 110 are desirably protected by an outer sheath or collar, and the lens cover 110 is secured by strong adhesive or flanges such that no rotating edges are exposed. In embodiments, the lens cover 110 can be shaped to provide additional optical function, for example, modify optical focal length, etc. Further, in various embodiments, the outer or exposed surface of the lens cover 110 is made hydrophobic and oleophobic. This can be accomplished by fine polishing of the surface, surface etching, a thin layer of hydrophobic coating material, for example, Teflon or parylene, or by other methods known in the art.

In various embodiments, the lens cover 110 is configured to rotate during operation, and through centripetal action, reduces impingement or build-up of debris or foreign matter on the endoscope 100 which impedes observation through the scope 104. The lens cover 110 is configured to rotate about an axis central to the cross-sectional circumference of the outer sheath body 102. In other words, the lens cover 110 includes a central rotation axis which is the same longitudinal axis passing through the central axis of the outer sheath 102 or the endoscope 100. In embodiments, the lens cover 110 is coupled to the outer sheath 102 of the endoscope 100, such as to provide stability with respect to the body of the scope, as well as providing a further seal against debris, in various embodiments. In embodiments, the lens cover 110 is coupled distally to the outer sheath 102 of the endoscope, i.e. the lens cover 110 is provided at the end of the endoscope 100. This allows the scope 104 to be provided with a maximized field of view, when the scope 104 is provided as close to the edge of the outer sheath 102 or end of the endoscope 100, such that any surrounding outer sheath structure does not block the peripheral vision of the scope 104. In various embodiments, the lens cover 110 is not provided at the end of the endoscope 100, to perhaps allow for the outer sheath 102, or an additional jacket coupled on the outer sheath 102, to further extend to perform such as a safety function in keeping any internal body matter from the rotating lens, cover 110.

In various embodiments, the lens cover 110 is coupled to a rotating lens shaft 112. The lens shaft 112 extends laterally and in parallel with the outer sheath 102 and is intended to be housed within the outer sheath 102 in the endoscope. The lens shaft 112 is thus provided for rotating motion in between the stationary scope 104 and outer sheath 102. In various embodiments, a lubricant 113 is provided within any one of the gap between the outer sheath 102 and the lens shaft 112, and the lens shaft 112 and the scope 104. The lubricant 113 provides maintenance of the operating gap between the respective shafts, and also acts against any friction or heat which may be caused by the rotating motion of the lens shaft against the scope 104 or the sheath 102. In embodiments, the lens shaft 112 is coupled by adhesive to the circumference of the lens cover 110. In embodiments, other coupled methods can be used, for example flanges, or nut and bolt, or cut and groove, etc. The lens shaft 112 is configured to rotate the lens cover 110 in accordance to a predetermined rotational speed.

In embodiments, the lens shaft 112 is driven by an actuation system 114 provided at a proximal end of the outer sheath 102 of the endoscope 100. Providing the actuation system 114 at the proximal end of the endoscope 100 allows for a slender and consistent form factor of the distal outer sheath as compared with standard endoscopes in the art. Such a slender form factor is critical, especially in consideration of certain endoscopy procedures such as laparoscopic surgery, where small incisions (usually 5-15 mm) are made to allow for an operation in the abdomen or pelvis or a patient.

In various embodiments, the actuation system 114 is a motor or a drill input mechanism 116. Further, the actuation system 114 includes a drive system 118, which includes drivetrains, shafts and connecting gears which cause the lens shaft 112 to be rotated in accordance to operational control.

In embodiments, the motor 116 is coupled to an input shaft or a drivetrain 120. The motor 116 is further coupled to a control system provided in a processor (not shown) providing supervisory control of the endoscope 100. Fine control, rotational speed and various other motor-related controls which affect the lens cover rotational function can be operated via the control system and the processor. The drive system 118 further includes an input gear 122 coupled onto the input shaft 120 and which rotates in accordance with the input shaft. The input gear 122 is then coupled teeth-wise with a lens shaft gear 124 which is coupled onto the lens shaft 112. The motor 116 thus actuates the drive system 118 which thereafter causes the lens shaft 112 to rotate accordingly. In other embodiments, additional gears can be provided in the drive system, for example to step up or down the rotation speed of the lens shaft 112, or to provide a further rotational or translational function for further features in the endoscope 100.

The lens shaft 112 is held in place at a proximal end of the outer sheath 102 with respect to the scope 104 and the outer sheath 102 with a lens shaft flange 126. By way of the lens shaft flange 126 and the coupling of the lens cover 110 to the outer sheath 102, the position of the lens shaft 112 is controlled with respect to the outer sheath 102, at least at the proximal and distal ends of the lens shaft 112.

In embodiments, the endoscope 100 includes a housing 130 which can include the optical imaging processing system, and can also include the actuation system 114 and the drive system 118 of the actuation system for enabling the rotation of the shield or lens cover or aperture cover 110 according to various embodiments. In embodiments, the housing 130 is provided at the proximal end of the outer sheath 102, i.e. the outer sheath 102 terminates at the housing 130. The outer sheath 102 and the housing 130 form a portion of the external body of the endoscope 100, and at least a mechanical actuation portion of the endoscope 100.

According to various embodiments, the scope 104 extends through a housing 110 and is further coupled to an optical imaging processing system proximal in comparison to the housing 110. A scope flange 108 can be provided prior to the entry of the scope 104 into the enclosure of the housing 110, and provides a coupling of the scope 104 for stability to the housing 110.

In various embodiments, the endoscope 100 can also be provided or be coupled with one or more of a light source, a camera system, an air insufflation port, singular or multiple surgical instruments or any other relevant or necessary apparatus for function.

In use according to various embodiments, the tip or the distal end of the endoscope 100 can be inserted into a subcutaneous space and manipulated or guided accordingly by a surgeon undertaking an MIS procedure, either through a direct manipulation of the outer sheath, or through other manipulation control.

During the MIS procedure, there is expected to be accumulation of debris on the tip or distal end of the endoscope 100, which may affect visualization through the scope 104. When an operating surgeon determines that there is impingement or build-up of debris adversely affecting vision during the MIS procedure, the actuation system 114 can be activated to operate the rotating lens cover 110. The lens cover 110, through centripetal or spinning action, rotates sufficiently such that any collected on or soiling the lens cover 110 is displaced and vision is restored to the endoscope 100. In various embodiments, the actuation system 114 is continuously in operation and the lens cover 110 is constantly rotated during the MIS procedure. Continually rotating the lens cover 110 takes a static surface which makes it more difficult for debris to impinge on the endoscope 100 in affecting visualization.

Accordingly, the speed of rotation of the lens cover 110 is an important consideration of the endoscope of the present disclosure. The rotational speed is configured in seeking to accomplish various objectives. Firstly, to prevent or remove debris sticking to the lens cover 110. Secondly, to break up larger debris particles such as fluid particles on the lens cover into smaller particles. Thirdly, the rotational speed of the lens cover should exceed the flicker fusion threshold of the human eye, which is about 15-60 Hz, thus rendering finer debris fouling invisible.

In various embodiments, the endoscope 100 of the present disclosure utilizes a rotating lens cover 110 configured to operate with a rotational speed selected from a range of between 500 rpm to 1000 rpm. In embodiments, the rotating lens cover 110 is configured to operate with a rotational speed of 1000 rpm. The inventors of the embodiments associated with present disclosure have established through empirical testing that a rotation speed of 1000 rpm can be sufficient to produce a flicker-free effect. It is also noted that balance is to taken in conjunction with the setting of the rotational speed of the lens cover. Although higher speeds will more effectively protect the lens due to the greater centrifugal forces, higher speeds may increase the theoretical risk of tissue injury.

FIG. 1B illustrates an exemplary embodiment of an endoscope 180 in demonstrating rotational speed calculation. The minimum speed required to produce a flicker free effect can be calculated as a function of obstruction size and distance from the center of the lens. In FIG. 1B, an obstruction of radius 1 mm is located 1 mm from the lens center. The following equations are used to determine the required rotational speed to achieve a flicker fusion threshold of 60 Hz.

$${x}^{2} = 2 {( x + y )}^{2} ( 1 - cos \frac{α}{2} ) \\ For x = 1 , y = 1 , \\ cos \frac{α}{2} = 1 - \frac{1}{8} ⇒ α = 58 ° \qquad ( 1 )$$

For a flicker threshold of 60 Hz, the length of obstruction= 1/60s=16.6 ms

### Rate of Rotation R Required:

$$\frac{58 °}{R} = 0.01666 ⇒ R = 3480 ° / s = 580 rpm$$

According to an embodiment, a lens cover rotational speed of 580 rpm is provided so as to meet the flicker fusion threshold of 60 Hz.

In various embodiments, the endoscope 100 terminates at its distal end with a circular edge perpendicular with the longitudinal central axis of the endoscope 100, i.e. with a zero degree angulation. The lens cover in this case is placed over the distal end of the outer sheath 102, and is similarly perpendicular with respect to the longitudinal central axis of the endoscope 100. In embodiments, the distal end of the endoscope can include an angled upward curved edge, or an angled distal end. A deviation from the perpendicular plane to the normal of the endoscope, as provided by an extension of the longitudinal central axis of the endoscope, provides an angulation of the endoscope. For example, the endoscope can be provided with angulations or views such as with a 0° angle, a 30° angle, a 45° angle or a 70° lens.

According to various embodiments, an endoscope with an angled distal tip is provided with certain modifications to the lens cover, or lens shaft, or the coupling of the lens cover to the outer sheath, to realize the provision of a visualization improvement system to allow for endoscopic operation with an endoscope with an angled distal tip. In various embodiments, suitable helical or worm gears and flexible couplings can be used to obtain rotation of the lens cover at a provided endoscopic angulation.

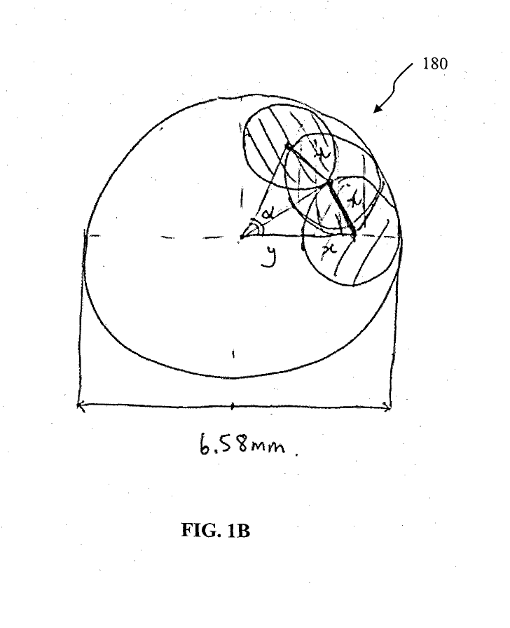

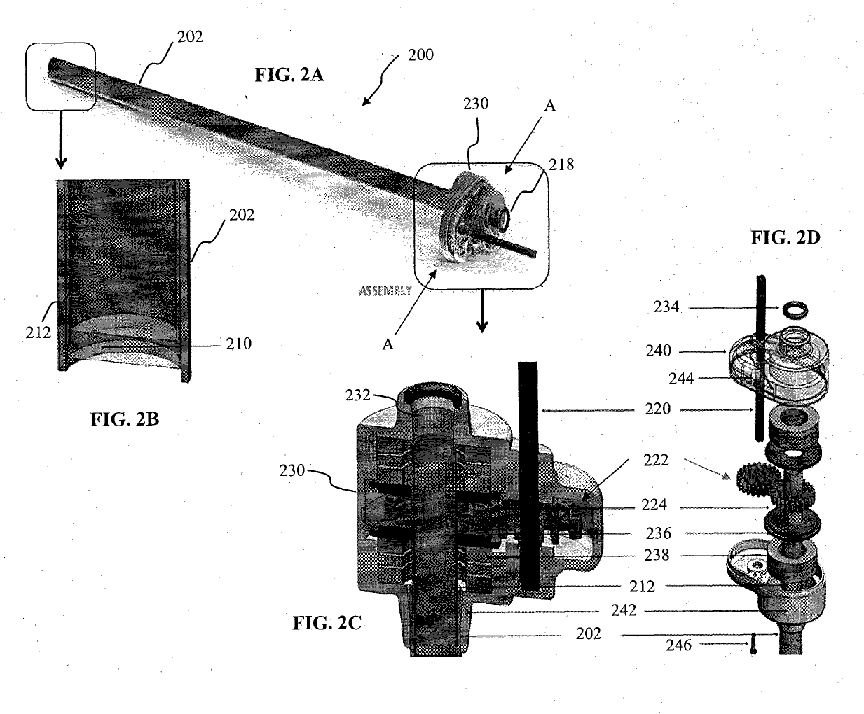

FIG. 2A illustrates a perspective view of a portion of the endoscope according to various embodiments. An endoscope 200 is shown, with a carrier body as provided by the external elongate outer sheath 202 and the housing 230, and without a scope or extension of an optical imaging system. The housing 230 is shown partially opened up, containing within a drive system 218 including a plurality of shafts, bearings and gears.

FIG. 2B illustrates a cross-sectional portion of the distal end of the endoscope of FIG. 2A. In embodiments, outer sheath 202 is provided, being the external housing of the elongate extension of the endoscope, arranged for retaining within an elongate scope or extension of an optical imaging system. The outer sheath 202 also includes within its internal housing a lens shaft 212, arranged to rotate about a central longitudinal axis with respect to the stationary outer sheath 202. The lens shaft 212 is coupled to a circumference of a lens cover 210, provided at the distal end of the endoscope 200 and the outer sheath 202.

According to various embodiments, the lens cover 210 is a machine acrylic lens, and is set into place with respect to the lens shaft 212 by way of adhesive. The lens cover 210 thus provides a protective shield for a scope to be housed within the outer sheath 202. The lens cover 210 provides protection from debris from a patient's body and exterior to the outer sheath, and thus keeps a housed scope free from debris. The lens cover 210 is further arranged to maintain visualization of the endoscope 200 during operation in a MIS procedure.

In embodiments, the lens shaft 212 is arranged to rotate, and as a result, cause the lens cover 210 to correspondingly rotate. In embodiments, the lens cover 210 is held in place at the distal end of the outer sheath by a coupling to the outer sheath. In such a way, the lens cover 210 does not deviate in position along the shaft of the outer sheath 202, which may lead to abutment on and damage to a housed scope. Rotation of the lens cover 210 leads to dispersal of any foreign debris settling on or fouling the lens cover 210 through centripetal action, which provides a maintaining of visualization with the endoscope 200.

FIG. 2C illustrates a blown up cross-sectional view of the housing of FIG. 2A. A cross-sectional view is provided along the line A-A of the housing 230. The view of the housing 230 is rotated, such that for perspective, the endoscope 200 extends distally down from the housing 230 and a received scope extends upwards, towards an optical imaging processing system. The input shaft 220 is also shown extending upwards toward a driving DC motor. FIG. 2D illustrates a blown up perspective assembly view with the components of the housing of FIG. 2C laid out.

Housing 230 is shown to be provided at a proximal end of the external outer sheath 202. Outer sheath 202 is illustrated to terminate at the housing 230. In embodiments, the outer sheath 202 is provided as a stainless steel outer sheath. In other embodiments, the outer sheath can include one or a plurality of other materials, which is suitable for insertion into a patient's body, without any detriment to the body or to the endoscope housed within the outer sheath. In other embodiments, the outer sheath can include a flexible tube or a flexible metal conduit which allows flexibility in operation.

The outer sheath 202 houses within its cylindrical body a lens shaft 210, arranged to rotate about the longitudinal central axis of the shaft 210. The lens shaft 210 is shown to be extending proximally past the termination of the outer sheath 202 into the housing 230. The lens shaft 210 is also shown to be terminated with respect to an enclosure 232 which is arranged to receive a scope and to retain the scope in the enclosure 232 and the housing 230. In embodiments, an O-ring 234 is provided for retaining a scope received in the endoscope housing 230. Alternatively, other securing mechanisms can also be used to retain a scope.

According to various embodiments, the lens shaft 210 is retained in the housing 230 and is coupled to a lens shaft gear 224. The lens shaft gear 224 is coupled around the circumference of the lens shaft 210 and is configured to move in accordance with an input shaft gear 222. The lens shaft gear 224 includes a plurality of teeth which are correspondingly fit to the teeth of the input shaft gear 222 such that rotational motion of the input shaft gear 222 will be translated to the lens shaft gear 224, and vice versa.

In embodiments, the input shaft gear 222 is retained within the housing 230 and is coupled to and about the circumference of an input shaft 220. The input shaft 220 extends out of the housing 230 and is coupled on the extended free end to a driving motor. The motor rotationally drives the input shaft, which in turn cranks the input shaft gear 222 and the lens shaft gear 224 and subsequently the lens shaft 212. The rotation of the lens shaft 212 causes the lens cover 210 to rotate, which removes any foreign debris fouling the lens cover 210. In various embodiments, the lens shaft 212 is included of brass, but other suitable metal can be used. In embodiments, the lens shaft 212 can be a flexible tube or metal conduit which allows the lens shaft 212 to flex according with the endoscope in operation, whilst still allowing for a rotational actuation of the lens cover 210.

In embodiments, to securely retain the lens shaft 212 within the housing 230 for optimum control and rotational motion translation through the gears, an inner shaft flange 236 is provided for retention about the cylindrical circumference of the lens shaft 212. In embodiments, the flange 236 can be stacked to provide a better hold and retention. Further, a thrust bearing 238 can be provided within the housing 230 to allow for smooth rotation of the lens shaft 212 about its central longitudinal axis. In embodiments, multiple bearings 238 can be stacked for higher efficiency.

In various embodiments, the housing 230 can include an upper enclosure portion 240 and a lower enclosure portion 242. The expressions upper and lower are provided with respect to the provided figures and only provide reference to the features of the embodiment. The lower enclosure 242 is arranged to receive and retain the proximal end of the outer sheath 202 and is directed towards the scope extension for insertion into a patient's body. The lower enclosure 242 of the housing 230 is arranged to receive the inner shaft flange 236 and the thrust bearings 238 so as to secure the lens shaft 212 for controlled rotation.

In various embodiments, the input shaft 220 extends out of the upper enclosure 240 of the housing 230. Further, the upper enclosure 240 also includes the enclosure 232 for receiving the scope and the O-ring 234 for securely retaining the scope. In embodiments, the upper enclosure 240 can also include a set of inner shaft flange and thrust bearings 238, such that the lens shaft 212 is well secured within the housing 230.

Having two sets of flanges and bearings in the upper and lower enclosures are useful as the upper enclosure 240 and the lower enclosure 242 can be mechanically joined and separated. This allows for ease of repair and maintenance. In embodiments, one of the housing enclosures, in this case, the upper enclosure 240, includes a threaded insert 244. The other housing enclosure includes a corresponding receptacle, which allows insertion of a screw 246 which retains both sides of the housing enclosures together. Other retaining arrangements of course can be possible.

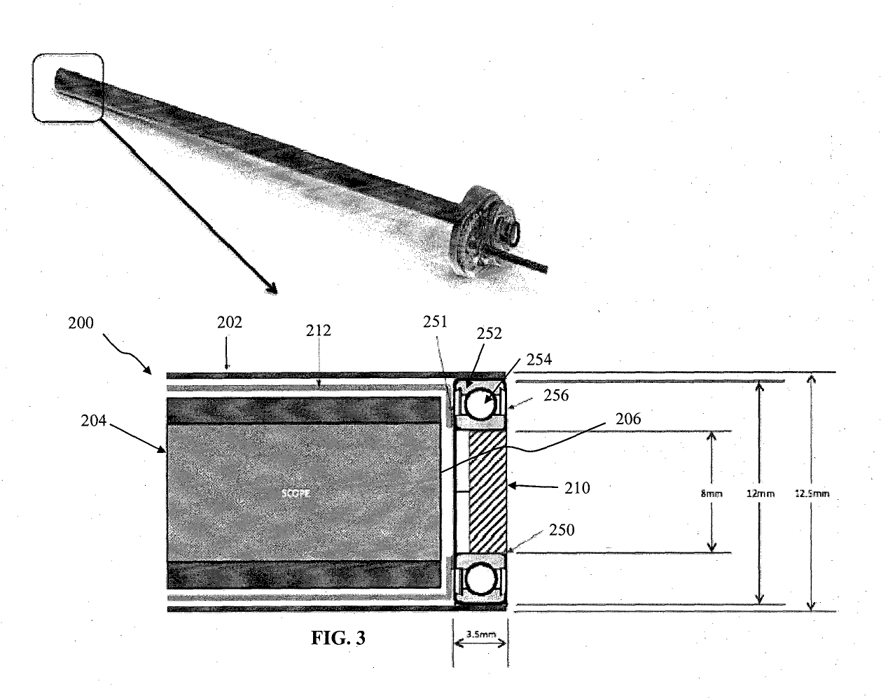

FIG. 3 illustrates a cross-sectional close-up view of a distal end of the endoscope of FIG. 2A. Endoscope 200 is shown to include a stationary outer sheath 202, a rotating lens shaft 212 provided within the cylindrical outer sheath 202, and configured to receive a scope 204 inner to the lens shaft 212. The scope 204 is intended to be stationary with respect to rotation about an axis. The lens shaft 212 is coupled to a lens cover 210 provided at the distal end of the endoscope 200. The scope 204 terminates at an optical aperture or an optical lens 206, and the lens cover 210 is provided as a shield for protection of the optical lens 206 from foreign debris during the MIS procedure.

In various embodiments, the lens cover 210 is also coupled to the distal end of the outer sheath 202 and is held in place at the distal end of the outer sheath 202. In embodiments, the lens shaft 212 is coupled to a retaining rim 250, which is thereafter coupled to the circumference of the lens cover 210. According to various embodiments, the lens shaft 212 is coupled to the retaining rim 250 with adhesive at 251. In other embodiments, the lens shaft 212 is coupled to the retaining rim 250 by way of brazing, or any other suitable methods for connection. In embodiments, the lens cover 210 is coupled on its circumference to the retaining rim 250, by way of adhesive, crimping, pressing, or any other suitable method. A corresponding receiving rim 252 is coupled on the inner surface of the distal end of the outer sheath 202.

A plurality of bearings 254 is provided between the retaining rim 250 and the receiving rim 252, and provides for the smooth rotation of the lens cover 210 in the endoscope 200. The provision of bearings 254 or micro-bearings provide a mechanical implementation of lubrication for the reduction of friction caused by the rotation of the lens cover 210. In embodiments, the bearings 254 are sealed 256 to provide consistency in performance. In other embodiments, the rotating surfaces of the lens cover with respect to the outer sheath can be made lubricious, with a friction-reducing coating of, for example, Teflon. In other embodiments, fluid lubrication can be used, with the provision of grease or oil. In other embodiments, a fluid cushion can be provided for reduction of friction. In such a case, a fluid actuation system can be provided at the proximal end of the endoscope, to provide for a fluid flow and a fluid cushion for acting at the rotating surfaces. In embodiments, the channel or gap between the outer sheath 202 and the lens shaft 212 may be slightly increased to provide for fluid channels for directing fluid flow. Advantageously, the endoscope according to various embodiments is provided with friction reducing features which achieve suitable lubrication and temperature control, which seeks to overcome the potential high frictional forces brought about by the rotating lens cover.

In other embodiments, the lens cover can be secured to the outer sheath with different mechanisms, the purpose of which is to allow operation without positional deviation or slippage of the lens cover.

In various embodiments, the endoscope 200 is a laparoscope for performing a MIS procedure through a small incision. One embodiment of the endoscope 200 is provided with dimensions suitable for use as a laparoscope, with the advantageous feature of providing a visualization improvement system of a rotating lens cover 210. The endoscope 200 is designed to retain a slim form factor, with the diameter of the endoscope 200 as determined by the diameter of the outer sheath 202, for insertion into a patient's body, being provided as 12.5 mm. The lens cover 210, including the rotation structure including the retaining rim 250, bearings 254 and receiving rim 252 have a diameter of 12 mm, and as can be noted, the rotation system is coupled directly to the internal surface of the outer sheath 202. Further, the exposed diameter of the lens cover 210 is provided as 8 mm. This allows a sufficient window for observation with the scope 206 through the lens cover 210. In embodiments, the thickness of the rotation system, as provided by the retaining and receiving rims 250, 252, is provided as 3.5 mm. Such a thickness allows for a suitable balance between protection of the optical lens or aperture of the scope 206 and providing a sufficient field of vision. It is of course noted that other dimensions are possible, in relation to the type and purpose of the endoscope, and any intended functions of the endoscope.

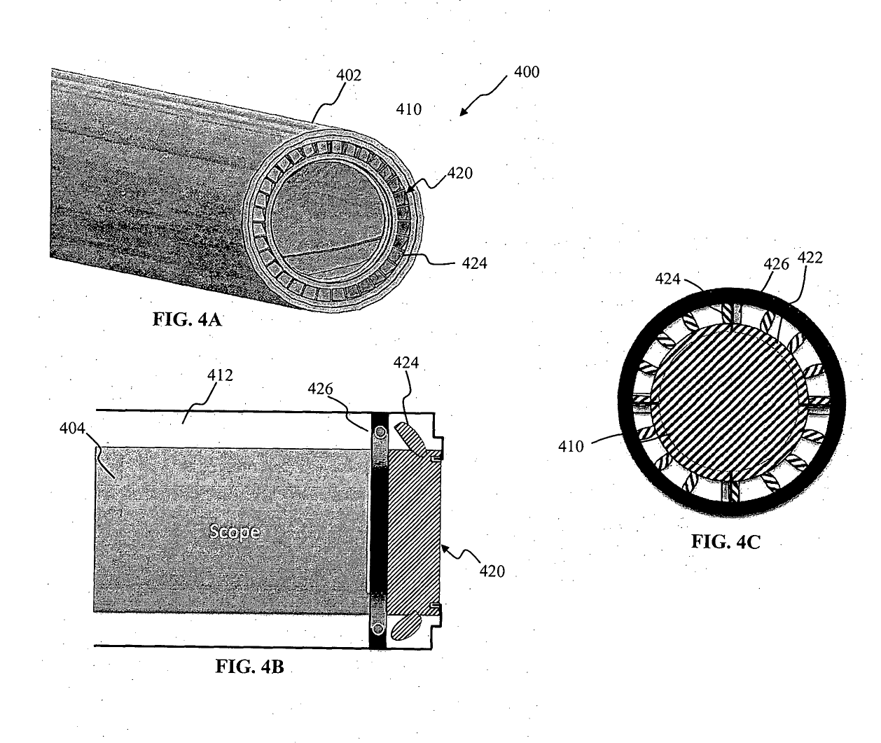

FIG. 4A illustrates an endoscope with a fluid flow actuation system according to an embodiment. In embodiments, endoscope 400 is shown with an outer sheath 402 and a lens cover 410 provided at the distal end of the endoscope 400. The lens cover 410 is provided perpendicular to the longitudinal central axis of the endoscope 400 and is for providing protection to an optical aperture or optical lens of a scope of an optical imaging system housed within the endoscope outer sheath 402.

In various embodiments, the torque for rotating the lens cover of an endoscope is provided with fluid flow. In an embodiment, the endoscope includes an actuation system (not shown) provided at a proximal end of the end of the endoscope and the outer sheath. In embodiments, the actuation system generates a fluid flow for the actuation of the lens cover for rotation. It is noted that the provision of a fluid flow for actuation allows for a reduction of mechanical moving parts, which would dramatically decrease frictional occurrences, as well as lubrication considerations.

The actuation system can be provided with a self-contained pressurized gas canister which is used to generate a pressurized gas flow to motivate the lens cover 410 into rotation. In embodiments, the actuation system is coupled to a pressurized gas delivery system for providing the fluid flow. In embodiments, the actuation system includes a pressurized gas delivered by a gas tank. In embodiments, the actuation system includes a fluid inlet and a regulator which can receive a pressurized gas delivery from a hospital wall supply or an external gas tank. In various embodiments, the fluid is carbon dioxide gas, but any other gas which is inert and harmless in quantities.

In various embodiments, the actuation system includes a suction system, and the fluid flow for generating a rotation is created by suction provided by the actuation system and generated in the proximal end of the endoscope. In embodiments, the actuation system includes a combination gas delivery and suction system. The fluid flow for generating a rotation is initiated by a pressurized fluid actuation from the gas delivery system. The fluid flow progresses from the proximal end of the endoscope to the distal end, where interaction with a rotation module coupled to the lens cover causes rotation of the lens cover. Further, the suction system creates a negative pressure at the proximal end and draws in fluid from the distal end of the endoscope. The suction system accentuates and improves the fluid flow, for more efficient rotation of the lens cover. Advantageously, providing the rotation module actuated by fluid flow removes the need to provide an additional electrical power source, as fluid delivery can be obtained from existing sources, such as the hospital wall supply.

FIG. 4B illustrates a cross-sectional view of the distal end of the endoscope of FIG. 4A, and can be used to illustrate the rotational actuation of the lens cover of the endoscope. In embodiments, the lens cover 410 is coupled to a rotation module 420, which is arranged for actuation by the actuation system such that the lens cover is rotated to improve visualization of the endoscope during a MIS procedure. The rotation module 420 is coupled to the circumference of the lens cover 410. Accordingly, the rotation module 420 includes a turbine ring 422, which includes a plurality of turbine blades 424 provided on the circumference of the rotation module 420. The turbine blades 424 are aerodynamically arranged to capture fluid flow from the proximal end of the endoscope 400, and accordingly rotate the lens cover 410 about the central longitudinal axis of the endoscope 400. It is further noted that the rotation module 420 includes a stator portion 426 coupled to the inner surface of the outer sheath 402, for holding the rotation module in place. The stator portion 426 also receives the turbine ring 422 and the lens cover 410, and is arranged to allow the turbine ring 422 to freely rotate about a central axis corresponding to the central cross-sectional point of the stator portion 426, and the central longitudinal axis of the endoscope 400.

FIG. 4C is a forward perspective view of the endoscope of FIG. 4A, and is useful in illustrating the structure of the rotating system. In embodiments, the fluid progresses from the proximal end of the endoscope 400 to the distal end of the endoscope through a channel 412 formed between the outer sheath 402 and the scope 404. The channel 412 can further include one or more guides, provided on the inner surface of the outer sheath 402, to direct fluid flow. In embodiments, the fluid flow is directed by the guides to a spiral motion. This allows the provision of a stator ring, and facilitates a more perpendicular angle of incidence of the fluid onto the turbine blades. This advantageously allows greater efficiency of power transfer from the fluid to the turbine ring.

The velocity of fluid flow and total surface area of the turbine blades 424 result in a theoretical maximum fluid power according to the equation:

Fluid Power=(0.5)ρAν³ (2)

where ρ is the fluid density, A is the surface area of the turbine blades and v is the fluid velocity. It is noted that power transfer would not be 100% efficient. Practical efficiency rates are likely to be 20-40%, and therefore the required velocity and surface areas will be empirically determined according to the endoscope embodiment.

In various embodiments, visualization of the endoscope during a MIS procedure is improved with the provision of a rotating lens cover driven by an actuation system provided at a proximal end of the endoscope. Further, additional systems or modules can also be incorporated into the endoscope to work in combination with the rotating lens cover. The present disclosure provides accordingly a plurality of other visualization improvement features for use in combination. In describing such additional modules, the present disclosure may or may not illustrate the additional modules in combination. According to various embodiments, any one of the visualization improvement features can also be provided on an endoscope without the rotating lens cover, to provide an endoscope with a standalone visualization improvement system. A combination of the visualization improvement features can also be incorporated as such.

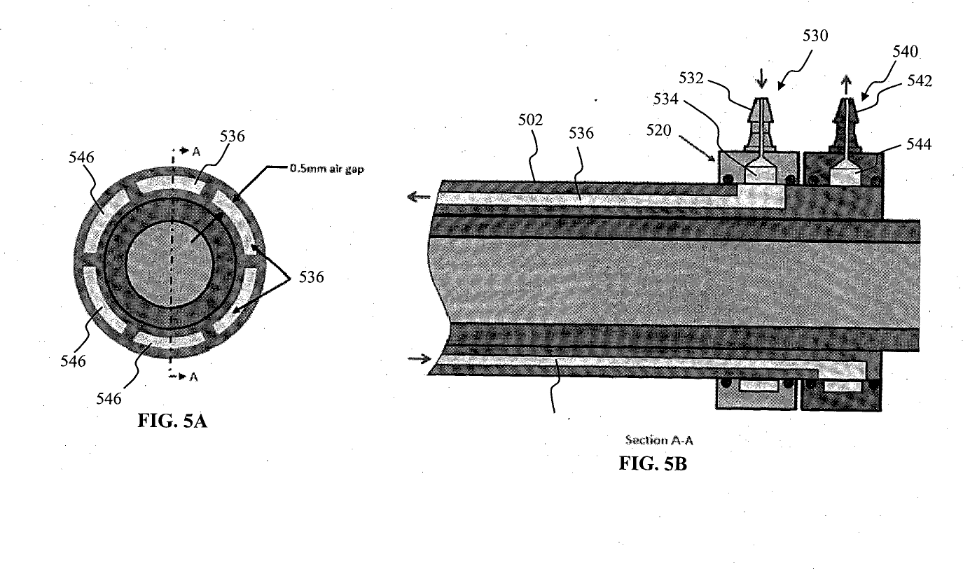

FIG. 5A illustrates a front view of an endoscope including a fluid flow feature for visualization improvement according to an embodiment. Endoscope 500 includes an outer sheath 502 enclosing within a scope 504 which is part of an optical imaging system for the endoscope 500. Endoscope 500 includes a fluid actuation system 520, which supports a visualization improvement feature for the scope 504. The fluid actuation system 520 is arranged to cooperate or work in combination with a rotating lens cover as described in earlier embodiments. In other embodiments, the fluid actuation system 520 for providing a fluid flow in moving debris may be utilized in the endoscope independent of the rotating lens cover.

FIG. 5B illustrates a cross-sectional view of the endoscope of FIG. 5A. In embodiments, the fluid actuation system 520 is provided at the proximal end of the endoscope 500 and particularly at the proximal end of the outer sheath 502. The actuation system 520 includes a fluid or gas inlet port 530 arranged to receive gas from a gas delivery module through a nozzle 532. The gas inlet port 530 and nozzle 532 includes a fluid delivery chamber 534 which leads into a fluid channel 536 defined along the length of the endoscope 500 to the distal end of the endoscope 500. In embodiments, the gas inlet port 530 is a manifold that bolts around the outer sheath 502 and over cross-cut openings in the fluid channel 536 in creating a gas-in port. In embodiments, the fluid channel 536 can be provided within the outer sheath 502, where the outer sheath is sufficiently wide such that a channel can be formed within the sheath. In other embodiments, an inner wall can be provided to the outer sheath 502, for forming the fluid channel 536.

According to various embodiments, the fluid actuation system 520 can include a gas outlet port 540. Gas outlet port 540 similarly includes a nozzle 542 for coupling to a suction or vacuum module for receiving fluid in use in the endoscope 500 for improving visualization. Gas outlet port 540 is a manifold that can be coupled around the outer sheath 520 and over an opening to a fluid channel 546. In embodiments, the fluid channel 536 for fluid delivered to the endoscope and the fluid channel 546 for receiving fluid from the endoscope are distinct and do not interact.

In embodiments, the actuation system 520 can include where the fluid chamber 534 of the gas fluid inlet port 530 is coupled to a plurality of fluid channels 536 for delivery of fluid to the distal end of the endoscope 500. The actuation system 520 also includes where the fluid chamber 544 of the gas outlet port 540 is coupled to a plurality of fluid channels 546 for receiving fluid from the distal end of the endoscope 500. As can be observed in FIG. 5A, according to various embodiments, three fluid channels 536 for delivery of fluid and three fluid channels 546 for receiving fluid are provided. The fluid channels 535, 546 open up at the distal end of the endoscope. In embodiments, the three fluid channels 536 for delivery of fluid are adjacent to each other and the three fluid channels 546 for receiving fluid are similarly adjacent. This provides an arrangement where fluid is delivered to the distal end on one side and fluid received from the other side, as such creating an air curtain over the external face of the scope 504. Such an air curtain can perform at least two functions—that of removing debris from the external face of the scope, as well as providing a buffer between the distal end of the endoscope and any impinging foreign debris. In other embodiments, the fluid channels are alternatively provided.

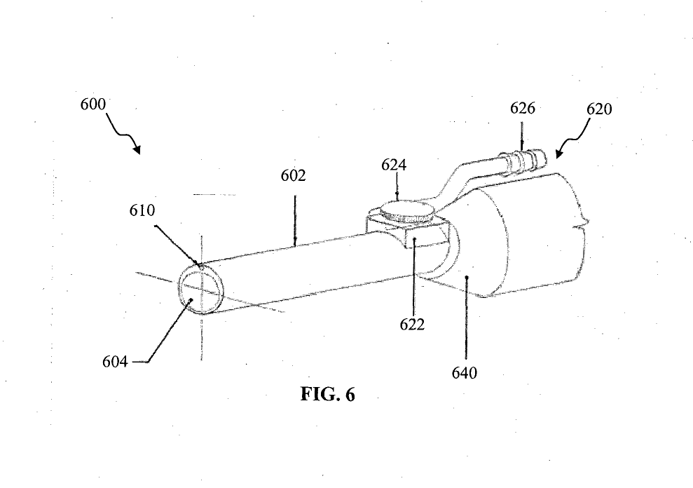

FIG. 6 illustrates a perspective view of an endoscope including a fluid column feature for visualization improvement according to an embodiment. FIG. 6 is not provided to scale and shows a proximal end and a distal end of an endoscope 600. Endoscope 600 includes an outer sheath 602 enclosing within a scope 604 which is part of an optical imaging system for the endoscope 600. Endoscope 600 includes a fluid actuation system 620, which supports a visualization improvement feature for the scope 604. Fluid actuation system 620 can be provided in or around a housing 640 at the proximal end of the endoscope.

Fluid actuation system 620 includes a regulator housing 622 and which is provided with a flow regulator dial or actuator 624. The regulator housing 622 is coupled to an air inlet port 626 which can be thereafter coupled to a gas delivery module or a gas insufflator machine or directly via a hospital wall supply. Fluid, and in most cases, compressed gas, is delivered through the air inlet port 626 and provided into the endoscope 600 through the regulator housing 622. The gas is provided to a fluid channel internal to the outer sheath 602, which directs the delivered gas from the inlet port to the distal end of the outer sheath 602. At the distal end of the endoscope 600 is provided with a gas column port, or an air jet port 610. The amount of gas output by the air jet port 610 can be controllable with the flow regulator dial 624, determining how strong or weak the air column output from the air jet port 610 is. The flow regulator 624 can also include a limiter to ensure that the fluid outlet pressure is within safety limits. Particularly, the flow and pressure of the air column is controlled such that the risk of causing air embolism is reduced.

The arrangement of the endoscope 600 according to various embodiments includes one or more fluid channels in close apposition to the endoscope optical channel, the fluid channel directing the fluid flow towards the surgical field with sufficient energy to displace oozing blood or other organic material, a fluid actuating element at the proximal end, and a flow regulating element to control the fluid pressure. In embodiments, the fluid used is carbon dioxide gas, but could also be saline, air etc. The system is built as part of the endoscope. In other embodiments, the system is integrated into a sheath that fits over the endoscope, with additional elements to secure the sheath to the endoscope.

According to various embodiments, the fluid column enables directed displacement of visual-impeding matter to facilitate visualization. Although it is envisioned that the fluid column will be most useful for displacement of blood or semi-solid blood clots, for exact location of points of bleeding, it is understood that the fluid column can also be used to displace other types of soft tissue or body fluids, in order to aid in visualization of underlying organ or tissue structures, for example critical nerves.

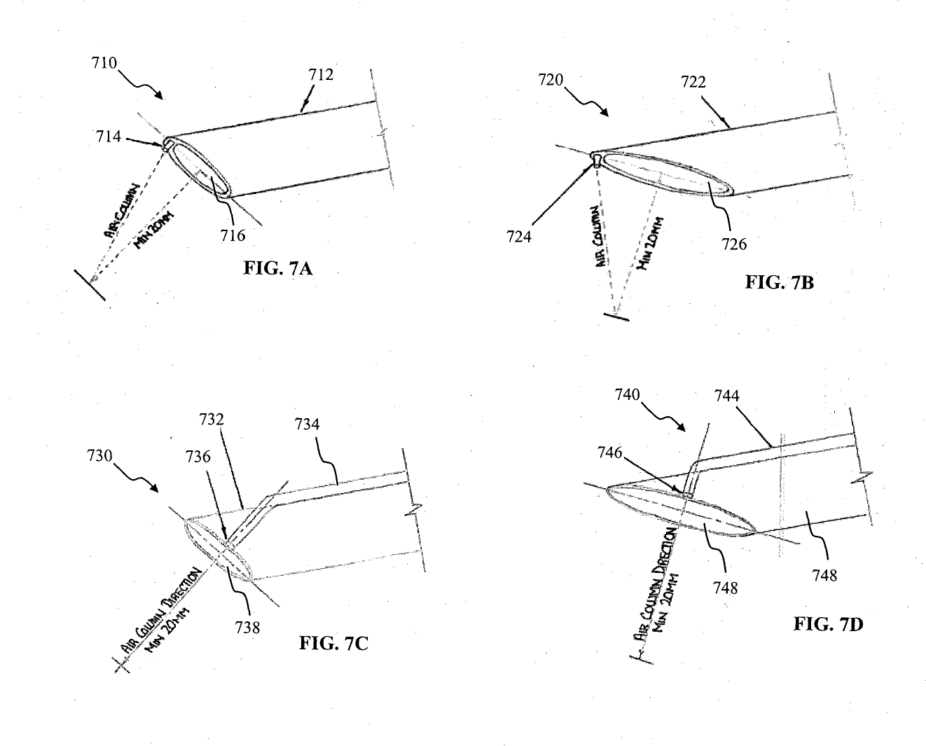

FIGS. 7A-7D illustrate endoscopes including a fluid column feature and in varying degrees of angulation according to various embodiments. FIG. 7A shows an endoscope 710 with a 30° angulation or view, and includes an outer sheath 712 which forms the elongate external body of the endoscope 710. The endoscope includes a fluid actuation system similar to that described for the embodiment above, and which terminates at an air column port 714 which protrudes from the distal end of the outer sheath 712 of the endoscope 710. The air column port 714 is provided at an off-center angle such that a column of air is directed to a predetermined distance directly normal and extending from the center of the lens aperture face of a scope 716 housed in the endoscope 710. In embodiments, the predetermined distance is at least 20 mm. In embodiments, the pressure of the air column is less than 10 mm Hg.

FIG. 7B shows an endoscope 720 with a 70° angulation or view, and includes an outer sheath 722 which forms the elongate external body of the endoscope 720. The endoscope includes a fluid actuation system similar to that described for the embodiment above, and which terminates at an air column port 724 which protrudes from the distal end of the outer sheath 722 of the endoscope 720. The air column port 724 is provided at an off-center angle such that a column of air is directed to a predetermined distance directly normal and extending from the center of the lens aperture face of a scope 726 housed in the endoscope 720. In embodiments, the predetermined distance is at least 20 mm. In embodiments, the pressure of the air column is less than 10 mm Hg.

FIG. 7C shows an endoscope 730 with a 30° angulation or view, and includes an outer sheath 732 which forms the elongate external body of the endoscope 730. The endoscope includes a fluid actuation system similar to that described for the embodiment above, and where the gas regulator feeds compressed air delivered from an external module into a fluid channel 734 provided external to the outer sheath 732 of the endoscope 730. The fluid channel 734 is directed along the length of the outer sheath 732 until near the distal end, where it is then angled and terminates at an air column port 734. The air column port 736 is thus provided adjacent and on the external surface of the outer sheath 732. The air column port 736 is provided at an off-center angle such that a column of air is directed to a predetermined distance directly normal and extending from the center of the lens aperture face of a scope 738 housed in the endoscope 730. In embodiments, the predetermined distance is at least 20 mm. In embodiments, the pressure of the air column is less than 10 mm Hg.

FIG. 7D shows an endoscope 740 with a 70° angulation or view, and includes an outer sheath 742 which forms the elongate external body of the endoscope 740. The endoscope includes a fluid actuation system similar to that described for the embodiment above, and where the gas regulator feeds compressed air delivered from an external module into a fluid channel 744 provided external to the outer sheath 742 of the endoscope 740. The fluid channel 744 is directed along the length of the outer sheath 742 until near the distal end, where it is then angled and terminates at an air column port 744. The air column port 746 is thus provided adjacent and on the external surface of the outer sheath 742. The air column port 746 is provided at an off-center angle such that a column of air is directed to a predetermined distance directly normal and extending from the center of the lens aperture face of a scope 748 housed in the endoscope 740. In embodiments, the predetermined distance is at least 20 mm. In embodiments, the pressure of the air column is less than 10 mm Hg.

It can be noted that there may be two important safety concerns associated with the use of this air column feature: barotrauma and gas embolism. The present disclosure provides that both of these concerns can be mitigated by controlling the direction and pressure of the fluid stream at the point of impact with the patient's tissues and blood vessels.

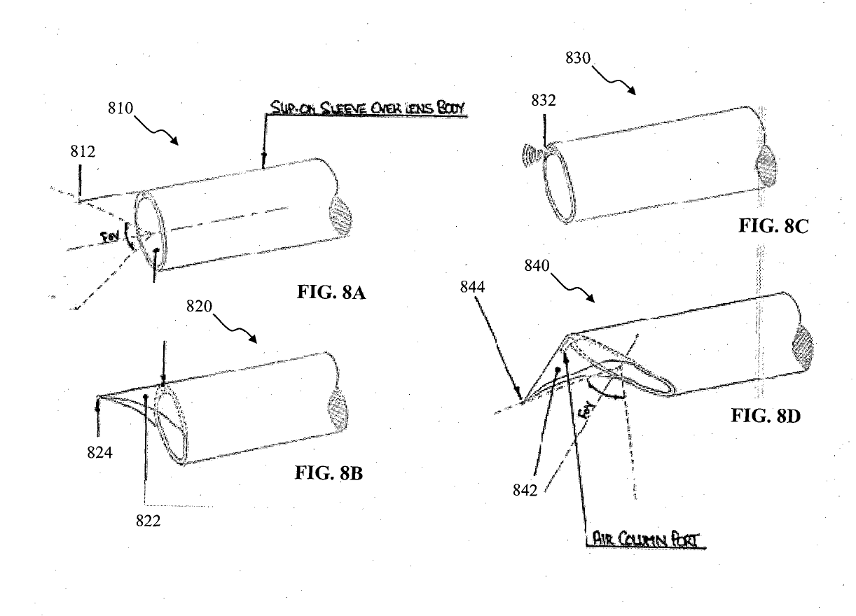

In embodiments, an additional safety feature is provided to impose a minimum safety distance between the fluid outlet and the nearest anatomical structure. This distance is preferably set at 3-5 mm, which is the minimum depth of field achievable by current state of the art surgical scopes. FIGS. 8A-8D illustrate an endoscope including a safety feature according to various embodiments. In FIG. 8A, a rod 812 is provided as a safety feature at the distal end of an endoscope 810, the rod 812 providing a distance indication with respect to the tip of the endoscope. According to various embodiments, the length of the rod 812 correlates to the field of view angle for which the endoscope operates.

In FIG. 8B, a hood 822 is provided as a safety feature at the distal end of an endoscope 820, the hood 822 providing a distance indication with respect to the tip of the endoscope. The hood 822 is a curved structure protruding from the external distal face of the endoscope 820 and tapers to a point 824, at which provides an indication for safety distance for use of the air column feature. Further, the hood 822 can provide against splashes on the observation aperture of the endoscope 822 from foreign debris. In other embodiments, any other physical extension of the sheath can be provided as a distance indicator to provide a safety distance for the air column.

In FIG. 8C, a proximity sensor 832 on the tip of the sheath is used to determine the distance of the nearest structure from the fluid outlet of the endoscope 830. The feedback from the proximity sensor 832 is processed as information in a processor at a proximal end and is conveyed to a visual display, either on the device or on-screen, or else conveyed via some alternative form of haptic feedback. In embodiments, alerts including any of variably pitched or timed audio tones, or force feedback can be incorporated.

In FIG. 8D, a hood 842 is provided as a safety feature at the distal end of an endoscope 840, the endoscope 840 being an endoscope with a 30° angulation. The hood 842 provides a distance indication with respect to the tip of the endoscope. The hood 842 is a curved structure protruding from the external distal face of the endoscope 840 and tapers to a point 844, at which provides an indication for safety distance for use of the air column feature. In other embodiments, any other physical extension of the sheath can be provided as a distance indicator to provide a safety distance for the air column.

In providing a fluid column feature for visualization improvement the risk of gas embolism is to be considered. Literature suggests that a net intracavity positive pressure of less than 20 mmHg above atmospheric pressure appears safe. CO2 is the most widely used insufflation gas as it is well-suited for creating a pneumoperitoneum because of its properties of being chemically inert, colorless, inexpensive, readily available and less combustible than air. CO2 is also highly water soluble which reduces its probability of causing embolism compared to a less soluble gas.

The likelihood of CO2 embolism occurring via insufflation depends on the balance between the volume of undissolved CO2 entering the enclosed body cavity and the amount of CO2 that is being removed. An excess of CO2 entering increases the likelihood of embolism. Direct insufflation into the vasculature will also produce emboli even if gas is removed from the body cavity.

Further, it is provided that parameters such as hydrational and volaemic state, cumulative intra-abdominal pressures above atmospheric, or excess pressures over insufflation pressures and/or intravascular pressures should also be considered in leading to using the endoscope of various embodiments safely and in consideration of air emboli.

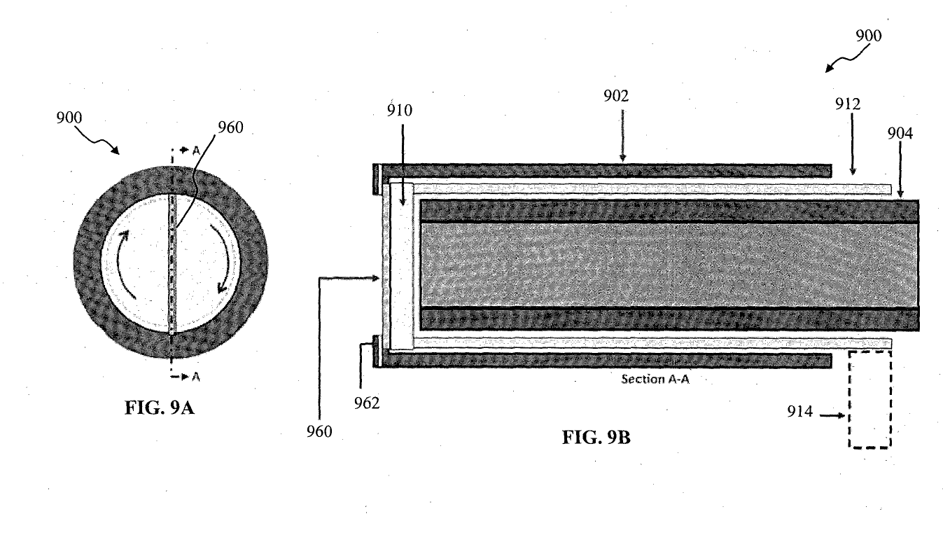

FIG. 9A illustrates a front view of an endoscope including a wiper element feature for visualization improvement according to an embodiment. Endoscope 900 includes an outer sheath 902 enclosing within a scope 904 which is part of an optical imaging system for the endoscope 900. Endoscope 900 includes a wiper element 960, which supports a visualization improvement feature for the scope 904. It is noted that the wiper element 960 is arranged for use in conjunction with a rotating lens cover 910. FIG. 9B illustrates a cross-sectional view of the endoscope of FIG. 9A.

In embodiments, the endoscope includes a lens shaft 912 coupled to the circumference of the lens cover 910 on one end and to an actuation system 914 for driving the lens shaft 912 on the other end. The lens cover 910 is actuated to rotate and thus disperse debris gathered on the external surface of the lens cover 910 and improving visualization.

The wiper element 960 is a single elongate element or a band coupled diametrically across the outer sheath 902. The wiper element 960 is intended to be a passive element arranged to abut the external surface of the lens cover 910. While the lens cover 910 rotates, the wiper element 960 provides a counteracting sweeping motion across the lens cover 910, thereby cleaning the lens cover and improving visualization of the endoscope 900. In embodiments, the outer sheath 902 can include a drainage channel 962 or multiple drainage channels through the outer sheath structure, thereby allowing debris accumulated by the sweeping wiper element 960 and propelled radially outward towards the outer sheath 902 to be expelled.

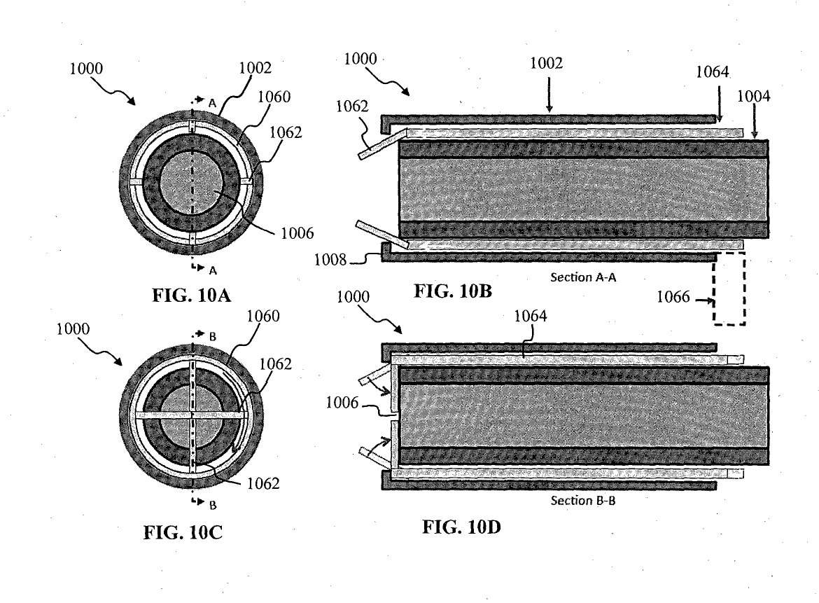

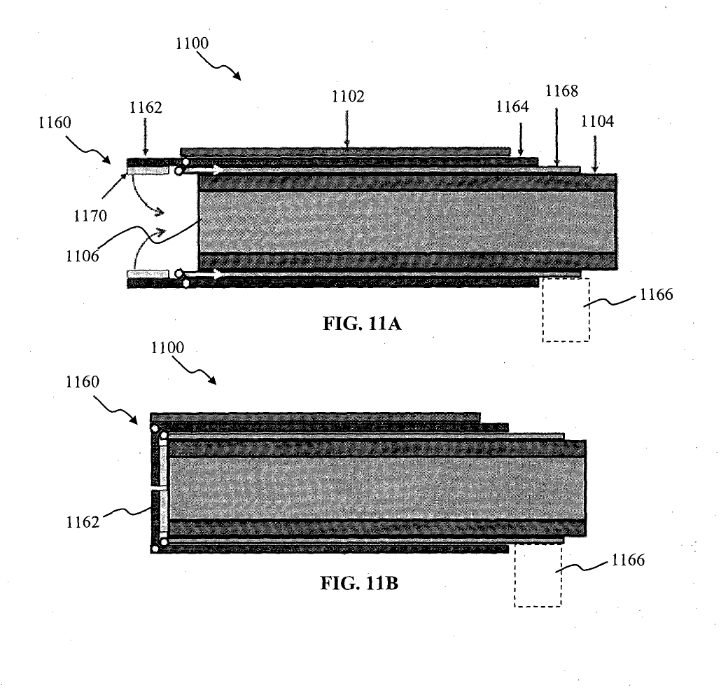

FIGS. 10A-10D illustrate an endoscope including a deployable wiper feature for visualization improvement according to an embodiment. FIG. 10A illustrates a front view of the endoscope with the deployable wiper 1060 in an unengaged position. FIG. 10B is a cross-sectional view of the endoscope with the deployable wiper in the unengaged position. Endoscope 1000 includes an outer sheath 1002 enclosing within a scope 1004 which is part of an optical imaging system for the endoscope 1000. Endoscope 1000 includes a deployable wiper 1060 which supports a visualization improvement feature for the scope 1004. The deployable wiper 1060 is arranged to cooperate or work in combination with a rotating lens cover as described in earlier embodiments. In other embodiments, the deployable wiper 1060 may be utilized in the endoscope independent of the rotating lens cover. In the figures, the deployable wiper is shown to operate without the rotating lens cover.

Deployable wiper 1060 includes at least one wiper element 1062 arranged to be raised from the external surface of the optical aperture or optical lens 1006 of the scope 1004 in an unengaged position. In embodiments, there are provided four wiper elements 1062, each wiper element set out with respect to quarterly points of the circumference of the outer sheath 1002. Other embodiments may include a different number of wiper elements. In the unengaged position, the wiper element 1062 is intended to be elevated such that observation with the scope 1004 is minimally impeded. In embodiments, the stationary outer sheath 1002 encloses internally the scope 1004 as like a sleeve or a sheath. The outer sheath 1002 includes tip retention portion 1008, which is an extension of the sheath cylindrically inward such that the scope 1004 is retained within the outer sheath 1002.

The wiper elements 1062 are coupled to a rotating inner cylinder 1064 provided in between the outer sheath 1002 and the scope 1004. The inner cylinder 1064 is coupled to an actuation system 1066 arranged to rotatably drive the inner cylinder 1064 through an electromagnetic motor and a corresponding gear system. Further, the actuation system 1066 includes a positional actuator which actuates the inner cylinder 1064 longitudinally bi-directionally towards and away from the distal end of the outer sheath 1002. The positional actuator also provides determination for the transition of the deployable wiper 1060 from the unengaged position to an engaged position.

In embodiments, the wiper elements 1062 are hingedly coupled to the inner cylinder 1064. In the unengaged position, the inner cylinder 1064 is drawn proximally by the positional actuator to a first retracted position such that the wiper elements 1062 are elevated above the optical aperture 1006 of the scope 1004. The wiper elements 1062 are elevated with respect to a fulcrum point provided by the edge of the scope 1004 due to the longitudinal proximal motion of the inner cylinder 1064. Further, the wiper elements 1062 are held in place in the unengaged position due to the counteracting position of the tip retention portion 1008.

FIG. 10C illustrates a front view of the endoscope with the deployable wiper 1060 in an engaged position. FIG. 10D is a cross-sectional view of the endoscope in the engaged position. In operation, the positional actuator provides a translation actuation of the inner cylinder 1064 distally to a second extended position, thereby causing the wiper elements 1062 to transition to an engaged position. In pushing the inner cylinder 1064 distally, the wiper elements 1062 move out of association with the fulcrum point provided by the edge of the scope 1004. Further, the inner cylinder 1064 is arranged to abut the inner surface of the tip retention portion 1008, which causes the hinged wiper elements 1062 to be urged towards the external surface of the optical aperture 1006 of the scope 1004. Rotation actuation is provided by the actuation system 1066, which allows the wiper elements 1062 to provide a sweeping motion in cleaning the external surface of the optical aperture 1006 in improving visualization of the endoscope 1000. Further, one or more drainage channels can be provided in the outer sheath 1002 for debris accumulated by the wiper elements 1062 to be expelled.

In various embodiments, the endoscope 1000 is provided with a rotating lens cover and the deployable wiper 1060. In embodiments, the inner cylinder 1064 is arranged to rotate with the same rotational axis as the rotating lens cover. In embodiments, the inner cylinder 1064 is arranged to rotate in the same direction as the rotating lens cover. In embodiments, the inner cylinder 1064 is arranged to rotate at a greater rotational speed as the rotational lens cover. In embodiments, the inner cylinder 1064 is arranged to rotate at a lower rotational speed as the rotational lens cover. In embodiments, the inner cylinder 1064 is arranged to rotate in the opposite direction as the rotating lens cover. In embodiments, a resting or pivot point of the wiper elements are provided in a lateral or inferior location, such that accumulated debris moving inferiorly due to gravity is not translated across the lens.

FIGS. 11A-11B illustrate an endoscope including a moving wiper feature for visualization improvement according to an embodiment. FIG. 11A illustrates a cross-sectional view of the endoscope with the moving wiper in an unengaged position. Endoscope 1100 includes an outer sheath 1102 enclosing within a scope 1104 which is part of an optical imaging system for the endoscope 1100. Endoscope 1100 includes a moving wiper 1160 which supports a visualization improvement feature for the scope 1104. The moving wiper 1160 is arranged to cooperate or work in combination with a rotating lens cover as described in earlier embodiments. In other embodiments, the moving wiper 1160 can be utilized in the endoscope independent of the rotating lens cover. In the figures, the moving wiper is shown to operate without the rotating lens cover.

Moving wiper 1160 includes at least one wiper element 1162 arranged to be raised from the external surface of the optical aperture or optical lens 1106 of the scope 1104 in an unengaged position. In embodiments, there are provided four wiper elements 1162, each wiper element set out with respect to quarterly points of the circumference of the outer sheath 1102. Other embodiments may include a different number of wiper elements. In the unengaged position, the wiper element 1162 is intended to be elevated such that observation with the scope 1104 is minimally impeded. In embodiments, the stationary outer sheath 1102 enclosed internally the scope 1104 as like a sleeve or a sheath.

The wiper elements 1162 are coupled to a rotating inner cylinder 1164 provided in between the outer sheath 1102 and the scope 1104. The inner cylinder 1164 is coupled to an actuation system 1166 arranged to rotatably drive the inner cylinder 1164 through an electromagnetic motor and a corresponding gear system. According to various embodiments, the wiper elements 1162 are hingedly coupled to the rotating inner cylinder 1164. The inner cylinder 1164 is also coupled to the outer sheath 1102 such that the inner cylinder 1164 does not displace longitudinally with respect to the outer sheath 1102. Accordingly, such a coupling includes a rotational function such that the inner cylinder 1164 can rotate with respect to the stationary outer sheath 1102. According to various embodiments, the wiper elements are further hingedly coupled to a wiper activation linkage 1168. The wiper activation linkage 1168 is further provided in between the inner cylinder and the scope 1004.

In embodiments, the actuation system 1066 includes a positional actuator which actuates the wiper activation linkage longitudinally bi-directionally towards and away from the distal end of the outer sheath 1102. The positional actuator also provides determination for the transition of the deployable wiper 1160 from the unengaged position to an engaged position. The wiper activation linkage 1168 is arranged to rotate correspondingly with the inner cylinder 1164.

In embodiments, the wiper elements 1162 are hingedly coupled to both the inner cylinder 1164 and the wiper activation linkage 1168. In the unengaged position, the wiper activation linkage 1168 is actuated distally to a first extended position by the positional actuator such that the wiper elements 1162 are elevated above the optical aperture 1106 of the scope 1104. According to various embodiments, the distal end of the wiper activation linkage 1168 abuts the rigid wiper elements 1162 and causes the wiper elements 1162 to be elevated with respect to the hinged pivot point of the coupling with the inner cylinder 1164. Further, the wiper elements 1162 are held in place in the unengaged position by the wiper activation linkage 1168 in the first extended position.

FIG. 11B is a cross-sectional view of the endoscope with the moving wiper in the engaged position. In operation, the positional actuator provides a translation actuation of the wiper actuation linkage 1168 proximally to a second retracted position, thereby causing the wiper elements 1162 to transition to an engaged position. In retracting the wiper actuation linkage 1168 proximally, the wiper elements 1162 are pulled into an engaged position where the wiper elements 1162 are in contact with the external face of the scope 1104.

In embodiments, the wiper elements 1162 can include a soft wiper portion 1170 providing on the inner surface of the wiper elements 1162, directed towards the external surface of the scope 1104. The soft wiper portion 1170 is designed to compress gently against the external surface of the optical aperture 1106 of the scope 1104 and providing a resilient contact with the external surface. Further, the soft wiper portion 1170 also provides adequate pressure on the external surface of the optical aperture to provide a scrub function which can remove more viscous debris from the optical aperture. Rotation actuation is provided by the actuation system 1166 on inner cylinder 1164, which allows the wiper elements 1162 to provide a sweeping motion in cleaning the external surface of the optical aperture 1106 in improving visualization of the endoscope 1100. Additionally, the inner cylinder 1164 can be arranged to rotate in a direction dissimilar to the direction of rotation of the lens cover. Further, one or more drainage channels can be provided in the outer sheath 1102 for debris accumulated by the wiper elements 1162 to be expelled.

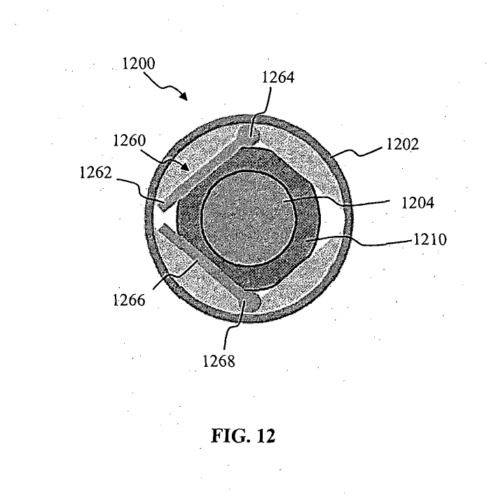

FIG. 12 illustrates a front view of an endoscope including a double wiper feature for visualization improvement according to an embodiment. Endoscope 1200 includes an outer sheath 1202 enclosing within a scope 1204 which is part of an optical imaging system for the endoscope 1200. Endoscope 1200 includes a wiper module 1260, which supports a visualization improvement feature for the scope 1204. It is noted that the wiper module 1260 is arranged for use in conjunction with a rotating lens cover 1210.

Wiper module 1260 is arranged to be provided over the external face of the rotating lens cover 1210. The wiper module 1260 further includes a first wiper element 1262 arranged to be pivoted at a circumference of the rotating lens cover 1210. The first wiper element 1262 is an elongate element arranged to be in sweeping contact with the rotating lens cover 1260, and to be rotatably pivoted about a pivot point 1264. The wiper element 1262 is arranged to carry out a sweeping motion covering an arcuate area which includes the external surface area of the lens cover 1210.

In embodiments, the wiper element 1262 is coupled to a rotating shaft at the pivot point 1264, the rotating shaft stretching proximally along the length of the outer sheath 1202 to a wiper element actuation system at the proximal end of the endoscope 1260. The wiper element 1262 is thus driven in operation by the wiper element actuation system. In other embodiments, the wiper element 1262 can be coupled to an electrically actuated pivoting element at the pivot point 1264. The electrically actuated pivoting element can be electrically coupled to a control module including a driving signal generator provided at the proximal end of the endoscope 1200. In other embodiments, the wiper element can be coupled to a hydraulically actuated pivoting element at the pivot point 1264. In embodiments the pivoting element at the pivot point 1264 can be a spindle.

According to various embodiments, the wiper module 1260 includes a second wiper element 1266 rotatably pivoted about a pivot point 1268. The second, wiper element 1266 is arranged to be pivoted at a circumference of the rotating lens cover 1210, diametrically opposite to the pivot point 1264 of the first wiper element 1262. The second wiper element 1266 is an elongate element arranged to be in sweeping contact with the rotating lens cover 1260, and to be rotatably pivoted about a pivot point 1268. The wiper element 1266 is arranged to carry out a sweeping motion covering an arcuate area which includes the external surface area of the lens cover 1210.

According to various embodiments, the second wiper element 1266 is provided with a rotation actuation system arranged to pivot the second wiper element 1266 to provide a sweeping motion over the lens cover, where the rotation actuation system for the second wiper element is similar to the rotation actuation system for the first wiper element. In other embodiments, the wiper module 1260 includes two different actuation systems.

According to various embodiments, the first and second wiper elements 1262 and 1266 are arranged to provide sweeping motions on the external face of the lens cover 1210. In embodiments, the sweep of the first wiper element 1262 in synchronization with the sweep of the second wiper element 1264. In embodiments, the first wiper 1262 is arranged to complete one sweep from one side to another of the arcuate area to be covered, before the second wiper 1264 begins its sweep. In such a way any accidental collision between the first and second wipers can be averted. Further, the first wiper 1262 and the second wiper 1264 can be arranged to sweep in opposing or alternate directions in each successive sweep.

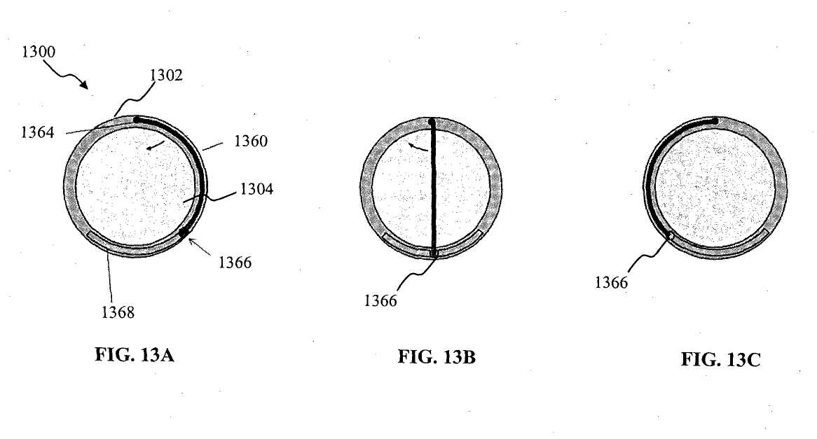

FIGS. 13A-13C illustrate an endoscope including a displaceable wiper feature for visualization improvement according to an embodiment. FIG. 13A illustrates a front view of an endoscope with a displaceable wiper in a first position. Endoscope 1300 includes an outer sheath 1302 enclosing within a scope 1304 which is part of an optical imaging system for the endoscope 1300. Endoscope 1300 includes a displaceable wiper 1360 which supports a visualization improvement feature for the scope 1304. The displaceable wiper 1360 is arranged to cooperate or work in combination with a rotating lens cover as described in earlier embodiments. In other embodiments, the displaceable wiper 1360 can be utilized in the endoscope independent of the rotating lens cover. In the figures, the displaceable wiper 1360 is shown to operate without the rotating lens cover.

Displaceable wiper 1360 includes a flexible wiper element 1362 coupled to an axle at a fixed pivot point 1364. In an embodiment, the flexible wiper element 1362 is retained on one end at a point on the circumference of the elongate outer sheath 1302. In an embodiment, the flexible wiper element 1362 is coupled at the other end to a carrier 1366 which is received in a guide track 1368. The carrier 1366 is arranged to guide the wiper element 1362 between predetermined operational positions.

In a first position, the flexible wiper element 1362 is displaced to the circumference of the outer sheath 1302. In an embodiment, the outer sheath 1302 includes an upraised groove for receiving and retaining the flexible wiper element 1362. In an embodiment, the flexible wiper element 1362 is a resilient element and in the first position, the flexible wiper element 1362 is at rest and in an unstretched condition. The first position of the carrier 1366 also corresponds to a right side limit of the guide track 1368.

FIG. 13B illustrates a front view of an endoscope with a displaceable wiper in a second position. Carrier 1366 is coupled to an actuation system which is arranged to drive the carrier from the first position to the second position. The actuation system can be electrically or mechanically driven. In the second position of the displaceable wiper module 1360, the flexible wiper element 1362 is provided diametrically opposed to the retaining point 1364. In other embodiments, the actuation system is coupled to the axis 1364, and the carrier 1366 moves passively along the guide track 1368.

In the second position, the resilient flexible wiper element 1362 is in a stretched condition and also provides sufficient tension such that the wiper element 1362 is effective for carrying out a sweeping and cleaning function. It is noted that in the second position, the stationary flexible wiper element 1362 can operate efficiently with respect to a rotating lens cover, the wiper element being in contact with the external surface of the lens cover. In embodiments, the flexible wiper element 1362 is arranged to be in contact with the optical aperture of the scope 1304 or the lens cover once the flexible wiper element departs from the outer sheath 1302.

FIG. 13C illustrates a front view of an endoscope with a displaceable wiper in a third position. According to various embodiments, the actuation system is configured to drive the carrier 1366 from the second position to the third position. The third position of the displaceable wiper also corresponds to a left side limit of the guide track 1368. In the third position, the flexible wiper element 1362 returns to an unstretched condition, and enters a position of rest in an upraised groove on the outer sheath 1302, which receives and retains the wiper element 1362.

The carrier 1366 is configured to be reversibly actuable back from the third position to the second position to the first position. According to various embodiments, by actuating the carrier 1366 and thus the flexible wiper element 1362 between positions, for example from the first position to the second position to the third position, the flexible wiper element 1362 carries out a sweeping motion and cleans off debris from the optical aperture or lens cover. Further, one or more drainage channels can be provided in the outer sheath 1302 for debris accumulated by the wiper element 1362 to be expelled. In other embodiments, the flexible wiper element, instead of being resilient, is retractably coupled to the pivot point 1364. This provides for a predetermined amount of tension to be applied to the flexible wiper with respect to the coupling at the other end to carrier 1366. The tension is applied through the positional translation by the carrier 1366, allowing the flexible wiper to carry out the sweeping function for improving visualization.

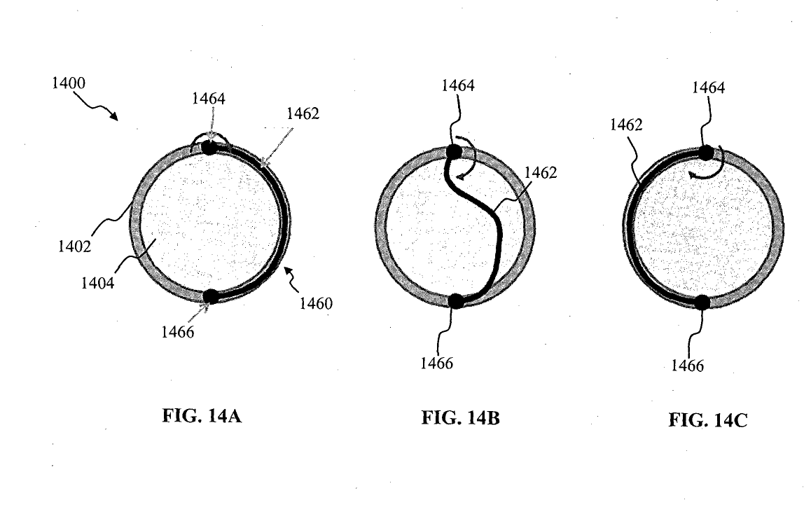

FIGS. 14A-14C illustrate an endoscope including a next displaceable wiper feature for visualization improvement according to an embodiment. FIG. 14A illustrates a front view of an endoscope with a displaceable wiper in a first position. Endoscope 1400 includes an outer sheath 1402 enclosing within a scope 1404 which is part of an optical imaging system for the endoscope 1400. Endoscope 1400 includes a displaceable wiper 1460 which supports a visualization improvement feature for the scope 1404. The displaceable wiper 1460 is arranged to cooperate or work in combination with a rotating lens cover as described in earlier embodiments. In other embodiments, the displaceable wiper 1460 can be utilized in the endoscope independent of the rotating lens cover. In the figures, the displaceable wiper 1460 is shown to operate without the rotating lens cover.

Displaceable wiper 1460 includes a resilient wiper element 1462 coupled to a first axle 1464 at a first pivot point. In an embodiment, the resilient wiper element 1462 is retained on one end at a point on the circumference of the elongate outer sheath 1402. In an embodiment, the resilient wiper element 1462 is coupled at the other end to a second axle. 1466 at a second pivot point. The resilient wiper element 1462 is retained at diametrically opposing points on the circumference of the outer sheath 1402. In embodiments, the first axle 1464 is arranged to be a driving axle, for actuation of the resilient wiper element 1462 from a first position to a second position. The second axle 1466 is arranged to be a passive axle. In embodiments, the first axle and the second axle are swivels and are rotatable about axes parallel to the length of the outer sheath 1402. It is noted that the resilient wiper element 1462 is arranged for motion only in the plane parallel to the external distal face of the endoscope 1400, which can also be considered normal to the longitudinal central axis of the endoscope 1400.

In a first position, the resilient wiper element 1462 is displaced to the circumference of the outer sheath 1402. In an embodiment, the outer sheath 1402 includes an upraised groove for receiving and retaining the resilient wiper element 1462. In an embodiment, in the first position, the resilient wiper element 1362 is at rest and in an unmodified condition. The resilient wiper element 1462 is arranged to elastically return the resilient element on a modification of its condition or shape.

In an embodiment, the first axle 1464 is coupled to the outer sheath 1402 and is driven by an actuation system to rotate from one side to the other. FIG. 14B illustrates a front view of an endoscope with a displaceable wiper in transition from a first position to a second position. As observed, when the first driving axle 1464 is rotated, the end of the resilient wiper element 1462 coupled to the driving axle 1464 moves along with the rotation of the axle. Accordingly, the resilient wiper element 1462 is displaced from rest on the outer sheath 1402 and comes into contact with the external face of the optical aperture of the scope 1404 or a lens cover, thus beginning a sweeping motion and clearing debris.

FIG. 14C illustrates a front view of an endoscope with a displaceable wiper at a second position after transition from a first position. In actuating the driving axle 1464 to the other side, the resilient wiper element 1462 is urged to carry out a sweep of the external face of the endoscope in returning to a position of rest on the other side of the outer sheath 1402 bisected by the diametrically opposing pivot points. Further, one or more drainage channels can be provided in the outer sheath 1402 for debris accumulated by the wiper element 1462 to be expelled.

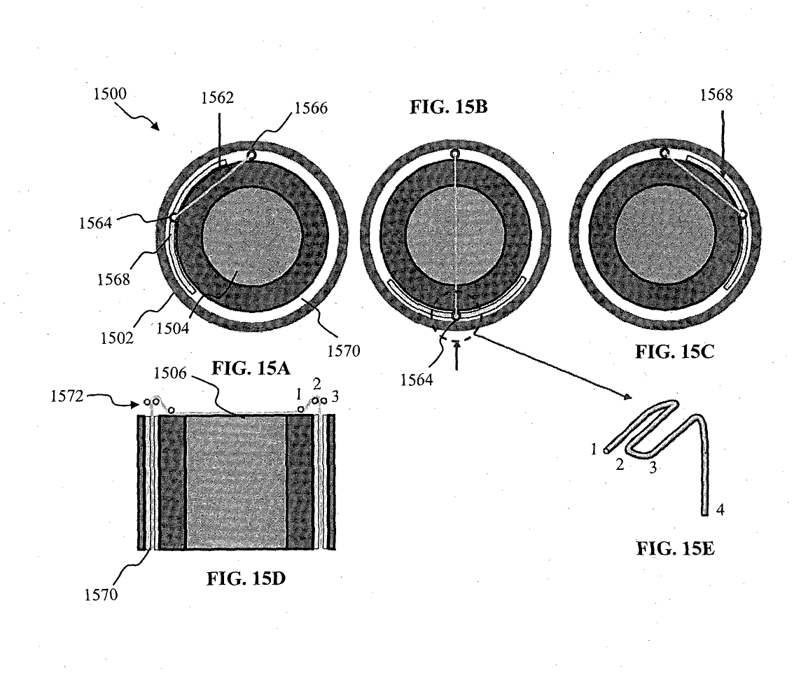

FIGS. 15A-15E illustrate an endoscope including a spoolable wiper feature for visualization improvement according to an embodiment. FIG. 15A illustrates a front view of an endoscope with a spoolable wiper in a first position. Endoscope 1500 includes an outer sheath 1502 enclosing within a scope 1504 which is part of an optical imaging system for the endoscope 1500. Endoscope 1500 includes a spoolable wiper 1560 which supports a visualization improvement feature for the scope 1504. The spoolable wiper 1560 is arranged to cooperate or work in combination with a rotating lens cover as described in earlier embodiments. In other embodiments, the spoolable wiper 1560 can be utilized in the endoscope independent of the rotating lens cover. In the figures, the spoolable wiper 1560 is shown to operate without the rotating lens cover.

Spoolable wiper 1560 includes a wiper element 1562 coupled at one end to a first spool port 1564 provided at a first point of the circumference of the outer sheath 1502, and coupled at another end to a second spool port 1566 provided at a second point of the circumference of the outer sheath 1502. In embodiments, the first spool port 1564 is coupled on a shuttle 1568 arranged to displace about the circumference of the outer sheath. In embodiments, the spoolable wiper 1560 is configured to be received in a spooling channel 1570 provided in between the outer sheath 1502 and the scope 1504. An actuation system for spooling and retaining excess provision of the wiper element can be provided at the proximal end of the endoscope to facilitate operation of the spoolable wiper 1560.

According to various embodiments, the second spool port 1566 is fixed in relation to its second point on the circumference of the outer sheath 1502. In embodiments, the first spool port 1564 is arranged to receive the spoolable wiper element 1562 and is coupled to a consolidation module of the actuation system in the proximal end, the consolidation module is arranged to draw in the spoolable wiper element 1562 and maintain a tension and drawing in any slack of the spoolable wiper element 1562. In embodiments, the second spool port 1566 is arranged to receive the wiper element 1562 and is coupled to a spool module of the actuation system in the proximal end, the spool model arranged to feed spoolable wiper element 1562 towards the distal end to provide new portion of spoolable wiper element upon request or design. In other embodiments, the first spool port 1564 feeds the wiper element, and the second spool port 1566 draws in the wiper element.

In embodiments, the shuttle 1568 is mechanically actuated and is arranged to circumferentially rotate the shuttle 1568 about the scope 1504. In embodiments, the shuttle 1568 is arranged to actuate towards one side of the endoscope 1500, as defined a line from the fixed spool port 1566 down to a diametrically opposing point of the outer sheath 1502 separating the endoscope into two sides. A first position of the spoolable wiper is provided when the shuttle 1568 is actuated to a left side limit. In embodiments, the shuttle 1568 is an elongate guide, and the spool port 1564 is provided in the middle of the elongate guide. A left arm of the elongate guide limits rotation of the shuttle 1568 when the left arm abuts the fixed spool port 1566, which can be referred to as a left side limit. Correspondingly, a right arm of the elongate guide limits rotation of the shuttle 1568 when the right arm abuts the fixed spool port 1566, which can be referred to as a right side limit.

In embodiments, the shuttle 1568 is actuated to circumferentially rotate the shuttle and the first spool port 1564 from a first position corresponding to a left side limit to a second position corresponding to a right side limit. FIG. 15B illustrates a front view of an endoscope with a spoolable wiper in transition from a first position to a second position. In FIG. 15B, the shuttle 1568 in transition provides for the first spool port 1564 to be diametrically opposing the second spool port 1566. It is noted that while during the transition from the first position to the second position, the spoolable wiper element 1562 is kept taut and is arranged to maintain contact with the external face of the scope 1504.

FIG. 15C illustrates a front view of an endoscope with a spoolable wiper in a second position. As the shuttle 1568 drags the spoolable wiper element 1562 across the lens surface of the scope during the transition from the first position to the second position, the spoolable wiper element 1562 carries out a function of accumulating and removing debris from the surface of the scope. In embodiments, the spoolable wiper element 1562 is a floss or a thin thread or a bundle or threads. In embodiments, the spoolable wiper element 1562 is designed to be highly absorbent for more efficient cleaning. Advantageously, a spooling actuation system provided in the proximal end of the endoscope allows for a new portion of the spoolable wiper element 1562 to be utilized at a predetermined time, or when the wiper element is deemed necessary for change.

In spooling, a consolidation module in the actuation system is controlled to drawn in a certain amount of the spoolable wiper element 1562, and for which the spooling module correspondingly allows for the amount to be drawn out from a spool. In such a way, the wiper element 1562 is exchanged, which provides for a continuous provision of clean wiper elements. In embodiments, the actuation system is configured to exchange a used portion for a new portion of wiper element after every sweep or transition from one point of the shuttle to the other.

FIG. 15D illustrates a cross-sectional view of an endoscope with a spoolable wiper. Wiper element 1562 is shown to be provided to and from a proximal spooling actuation system in a spooling channel 1570. Further, a retention module 1572 is provided on the distal end of the endoscope 1500, the retention module 1572 configured to receive a spoolable wiper element 1562 from a proximal end and causes the spoolable wiper element 1562 to maintain contact with the optical aperture 1506 of the scope 1504.

FIG. 15E illustrates a retention module provided for use with the endoscope of FIG. 15D. The module has 4 prongs, 3 that receive the wiper element, and one that enables the module to be secured to a portion of the endoscope or endoscope sheath. In an embodiment, the wiper is threaded through the prongs as illustrated in FIG. 5D. These prongs have smooth rounded surfaces to allow for smooth spooling of the wiper element, yet secure the wiper in close contact with the lens or lens cover of the endoscope, to enable efficient cleaning of the lens surface.

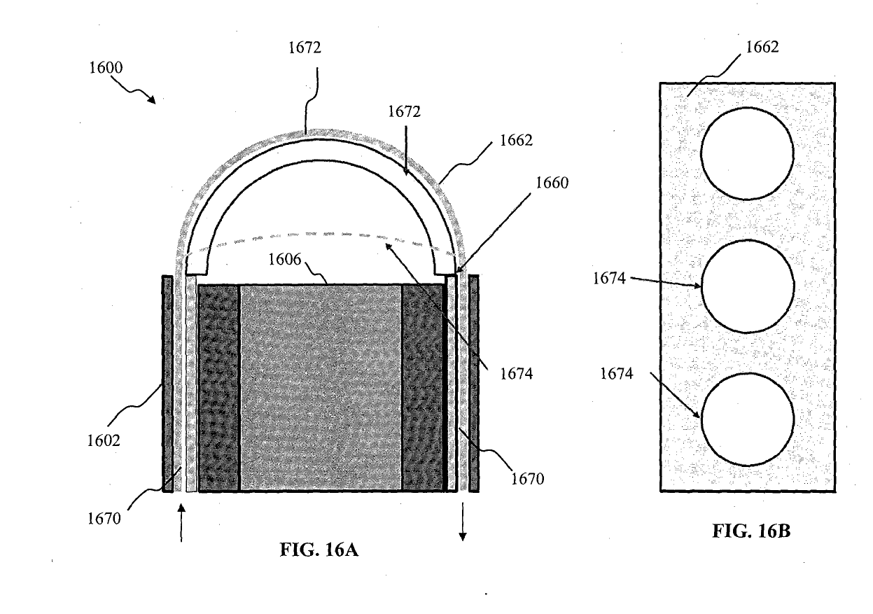

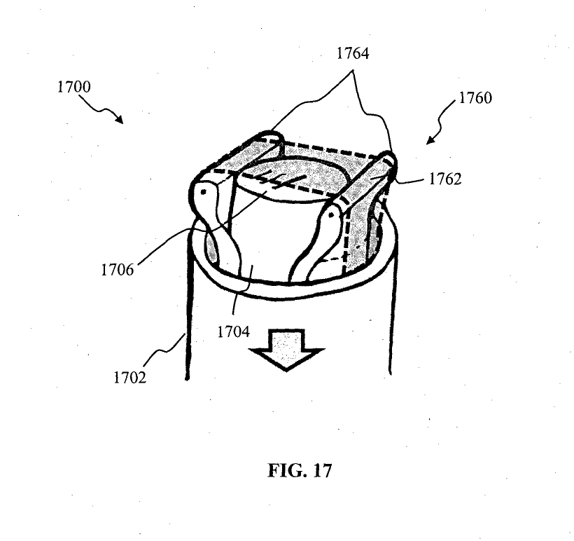

FIG. 16A illustrates a cross-sectional view of an endoscope including a next spoolable wiper feature for visualization improvement according to an embodiment. Endoscope 1600 includes an outer sheath 1602 enclosing within a scope 1604 which is part of an optical imaging system for the endoscope 1600. Endoscope 1600 includes a spoolable wiper 1660 which supports a visualization improvement feature for the scope 1604. The spoolable wiper 1660 is arranged to cooperate or work in combination with a rotating lens cover as described in earlier embodiments. In other embodiments, the spoolable wiper 1660 can be utilized in the endoscope independent of the rotating lens cover. In the figures, the spoolable wiper 1660 is shown to operate without the rotating lens cover.

Spoolable wiper 1660 includes a wiper element 1662 coupled to a spooling actuation system provided at a proximal end of the endoscope 1600. In embodiments, the wiper element 1662 is a broad strip and is arranged to clean an optical aperture or optical lens 1606 of the scope 1604. The strip needs to be flexible yet strong enough to withstand the frictional forces during spooling. In embodiments, the broad strip could include a woven material such as cotton gauze, and/or be reinforced on the top surface with tape. The tape reinforcement allows for fluid absorption while strengthening the strip against tearing during spooling. In embodiments, the broad strip can include holes or apertures to allow for unimpeded observation with the scope while the broad strip is continually spooled over the optical aperture 1606. In various embodiments, the wiper element 1662 is a clear flexible material for protecting the optical aperture of the scope. Such an arrangement can be considered a spoolable shield feature. When the material over the optical aperture is fouled, an adjacent clean section of strip is advanced across the lens, and the fouled section is withdrawn into the outer sheath.

The spooling actuation system allows for the wiper element 1662 to be spooled such that the wiper element 1662 is refreshed for effective cleaning. The spooling actuation system draws in the wiper element 1662 on one end and correspondingly releases the wiper element on the other end, through a spooling channel 1670 provided in between the outer sheath 1602 and the scope 1604.

Running a broad strip of material along and across the circular surface of a cylinder (such as a laparoscope) usually results in the highest tension forces being generated in the middle of the strip. This would mean that the sides of the optical lens 1606 are less effectively cleaned. In embodiments, this effect is mitigated with a highly absorbent cleaning material such as cotton or paper. In embodiments, a domed lens 1672 is provided over the optical aperture 1606 to mitigate such effect. Advantageously, the domed lens 1672 is arranged such that the dome lens does not affect observation with the scope 1604. Further, the dome lens is arranged over the scope 1604 at the distal end of the endoscope 1600 and allows for a smooth transition for the spoolable wiper element 1662 in and out of the spooling channel 1670, which evens tension on the wiper element 1662. FIG. 16B illustrates a spoolable wiper element according to an embodiment.

In embodiments, the wiper element 1662 includes a plurality of holes 1674, which allows for the wiper element to sit low on the domed surface 1672. Upon spooling, the force required for the wiper element 1662 to overcome in motion due to the dome sitting in the hole 1674 provides for greater tension at the sides of the holes during spooling, and may create a more even tension distribution and cleaning efficacy. In embodiments, a dome height of about 3 mm is provided for a 10 mm scope. Such an arrangement allows for smooth spooling, due to a more gradual curvature of the strip as compared with the abrupt 90 degree angles without the dome, and a good cleaning coverage of the sides of the lens.

FIG. 17 illustrates an endoscope including a spoolable wiper feature including extendable arms for visualization improvement according to an embodiment. Endoscope 1700 includes an outer sheath 1702 enclosing within a scope 1704 which is part of an optical imaging system for the endoscope 1700. Endoscope 1700 includes a spoolable wiper 1760 which supports a visualization improvement feature for the scope 1704. The spoolable wiper 1760 is arranged to cooperate or work in combination with a rotating lens cover as described in earlier embodiments. In other embodiments, the spoolable wiper 1760 can be utilized in the endoscope independent of the rotating lens cover. In the figures, the spoolable wiper 1760 is shown to operate without the rotating lens cover.

According to various embodiments, the spoolable wiper 1760 includes a pair of arms 1764, the arms 1764 being relatively extendable and retractable with respect to the outer sheath 1702. The arms 1764 arranged to be contained within the outer sheath 1702 of the endoscope. The arms 1764 are arranged to receive and retain a spoolable and elongated wiper element 1762 which is coupled at both ends to a spooling actuation system provided at a proximal end of the endoscope 1700. In embodiments, the wiper element 1762 is a broad strip that entirely covers an optical aperture 1706 of the scope 1704 when overlaid on the aperture 1706. According to various embodiments, the wiper element 1762 is configured to spool or advance such that a clean portion of the wiper element 1762 is provided to replace a fouled portion.

According to various embodiments, a distal portion of the outer sheath 1702 is arranged to be retractable. In a first rest position, the outer sheath 1702 is unretracted and houses the various features and functions of the endoscope 1700 within the outer sheath. In a second retracted position, the outer sheath 1702 is retracted and exposes the arms 1764, supporting a portion of the spoolable wiper element 1762 and the distal end of the scope 1704. In embodiments, in the retracted position, the arms 1764 are arranged to flare open and flatten out. In such a case, the wiper element 1762 is also allowed to rest on and contact the external surface of the optical aperture 1706.

In embodiments, the spooling actuation system is configured to carry out a spooling action of the wiper element 1762 when the outer sheath 1702 is in the retracted position, and the wiper element 1762 is resting on the external surface of the optical aperture 1706. Spooling while the wiper element 1762 is in contact with the optical aperture allows an efficient cleaning of the optical aperture 1706. In various embodiments, the wiper element 1762 is a broad strip. In embodiments, the wiper element 1762 is a tape with intermittent clear portions and cleaning portions.

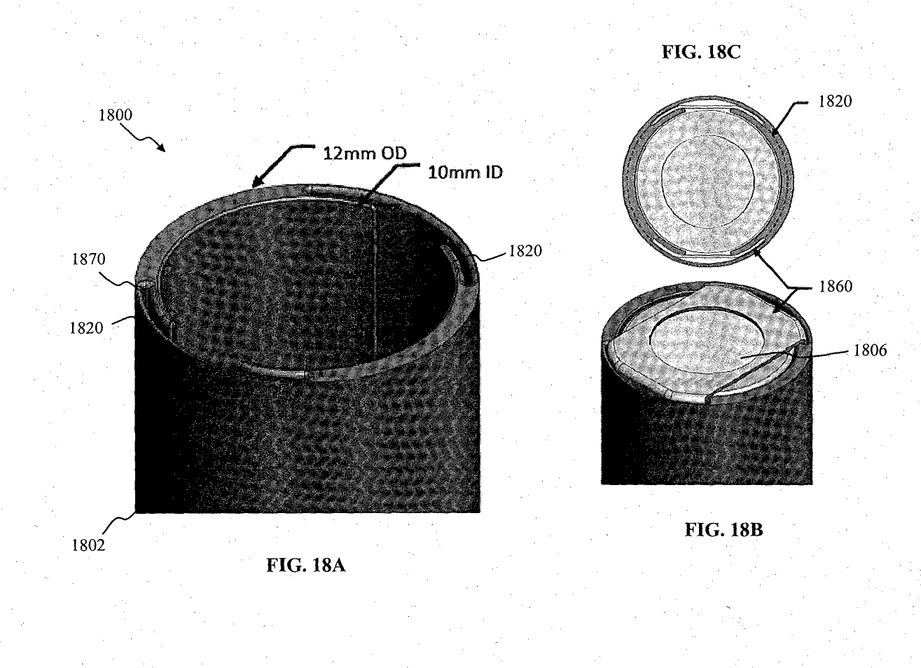

FIGS. 18A-18C illustrate an endoscope including a spoolable wiper feature including a tapered sheath guide for visualization improvement according to an embodiment. Endoscope 1800 includes an outer sheath 1802 enclosing within a scope 1804 which is part of an optical imaging system for the endoscope 1800. Endoscope 1800 includes a spoolable wiper 1860 which supports a visualization improvement feature for the scope 1804. The spoolable wiper 1860 is arranged to cooperate or work in combination with a rotating lens cover as described in earlier embodiments. In other embodiments, the spoolable wiper 1860 can be utilized in the endoscope independent of the rotating lens cover. In the figures; the spoolable wiper 1860 is shown to operate without the rotating lens cover.

FIG. 18A illustrates a cross-sectional perspective view of an outer sheath according to an embodiment. The outer sheath 1802 includes a set of guides 1820 through which a spoolable wiper element 1862 is received to and from the proximal end of the endoscope 1800. The spoolable wiper element 1862 is coupled to a spooling actuation system provided in the proximal end of the endoscope. In embodiments, the set of guides 1820 is formed by cut-outs provided in the outer sheath 1802. In embodiments, the set of guides 1820 is formed within the outer sheath 1802 structure. According to an embodiment, in such an instance the outer diameter of the outer sheath can be 12 mm, while the inner diameter of the outer sheath is 10 mm, including the set of guides provided in the outer sheath structure. In embodiments, the set of guides 1820 is provided by assembling a guide supplement onto the inner surface of the outer sheath 1802.

According to various embodiments, the set of guides 1820 provide an intermediate structure which changes the points and angle of inflection of the wiper element 1862 in transiting to and from a spooling channel 1870 perpendicularly onto the external face of the optical aperture 1806. In embodiments, the wiper element 1862 is an elongate rectangular broad strip, which has a breadth sufficiently large to overlay the surface area of the optical aperture 1806, to provide cleaning of the entire optical aperture surface. However, with such a breadth, the sides of the wiper element 1862 may not be able to clean effectively as the main inflection point can be identified towards the center of the strip. Advantageously, providing such a set of guides 1820 allows the provision of greater tension on both sides of the scope 1804, such that there is provided effective cleaning of the optical aperture.

FIG. 18B is a cross-sectional perspective view of the endoscope of FIG. 18A, further showing a spooling wiper. FIG. 18C is a top down view of the endoscope of FIG. 18B. In various embodiments, the wiper element 1862 is a broad strip. In embodiments, the wiper element 1862 is a tape with intermittent clear portions and cleaning portions. In, embodiments, the wiper element 1862 can include holes or apertures to allow for unimpeded observation with the scope while the broad strip is continually spooled over the optical aperture.

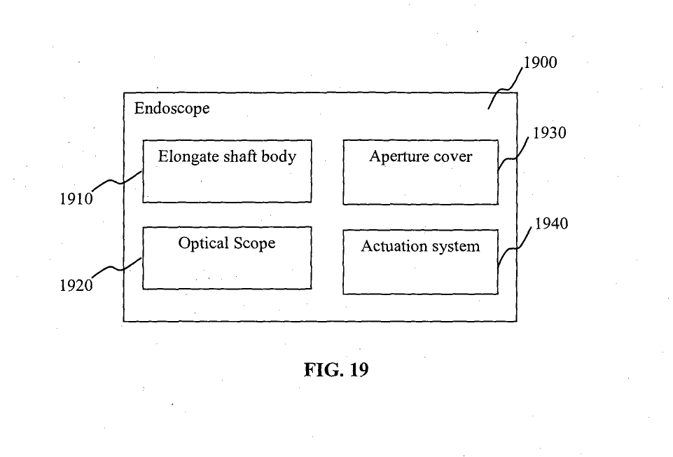

FIG. 19 illustrates a block schematic of an endoscope according to an embodiment of the present disclosure. According to an embodiment, there is provided an endoscope 1900. The endoscope 1900 includes an elongate shaft body 1910, and an optical scope 1920 provided within the elongate shaft body, the optical scope including an optical aperture provided at the distal end of the optical scope, corresponding to a distal end of the elongate shaft body. The endoscope 1900 further includes an aperture cover 1930 provided at the distal end of the elongate shaft body for shielding the optical aperture from debris external to the elongate shaft body, and an actuation system 1940 provided at a proximal end of the elongate shaft body. According to various embodiments, the actuation system drives the aperture cover to rotate.

In an embodiment, the actuation system includes a motor at a proximal end of the elongate shaft body.

In an embodiment, the motor drives a rotating shaft coupled to the circumference of the aperture cover.

In an embodiment, the rotating shaft is provided between the elongate shaft body and the optical scope.

In an embodiment, the aperture cover is coupled to the elongate shaft body through a plurality of bearings.

In an embodiment, the actuation system is a fluid flow actuation system.

In an embodiment, the actuation system includes any one of a pressurized gas delivery system and a suction system. In an embodiment, the actuation system can include a pressurized gas delivery system. In an embodiment, the actuation system can include a suction system.

In an embodiment, the actuation system includes a pressurized gas delivery system or a suction system.

In an embodiment, the aperture cover is circumferentially coupled to a turbine ring including a plurality of blades.

In an embodiment, an inner surface of the elongate shaft body includes a plurality of fluid guides for directing fluid onto the turbine ring blades.

In an embodiment, the aperture cover is coated with any one of a hydrophobic coating or an oleophobic coating. In an embodiment, the aperture cover is coated with a hydrophobic coating. In an embodiment, the aperture cover is coated with an oleophobic coating.

In an embodiment, the endoscope further includes any one of a helical gear or a worm gear for angulated operation of the aperture cover. In an embodiment, the endoscope further includes a helical gear. In an embodiment, the endoscope further includes a worm gear.

In an embodiment, the aperture cover is configured to operate at a rotation speed which exceeds the flicker fusion threshold.

In an embodiment, the rotation speed is selected from a range of between 500 rpm to 10,000 rpm.

In an embodiment, the endoscope further includes a secondary cleaning module coupled to the distal end of the elongate shaft body, for cooperation with the aperture cover for improving visualization.

In an embodiment, the secondary cleaning module is a fluid flow actuator arranged to provide a fluid flow over an external surface of the aperture cover.

In an embodiment, the fluid flow actuator is coupled to a fluid port on a distal tip on the elongate shaft body.

In an embodiment, the fluid port is coupled to a fluid channel provided in the elongate shaft body.

In an embodiment, the fluid port is directed to displace debris from in front of the external surface of the aperture cover with a fluid flow.

In an embodiment, the endoscope further includes a safety mechanism to indicate a safety distance from the fluid port.

In an embodiment, the safety mechanism is any one of a protruding rod or a protruding hood. In an embodiment, the safety mechanism is a protruding rod. In an embodiment, the safety mechanism is a protruding hood.

In an embodiment, the safety mechanism is a proximity sensor.

In an embodiment, the proximity sensor is arranged to provide a feedback indication to a user.

In an embodiment, the secondary cleaning module is a fluid flow circulator arranged to provide a fluid flow circulation over an external surface of the aperture cover.

In an embodiment, the fluid flow circulator includes a gas inlet port and a gas outlet port provided at the proximal end of the elongate shaft body.

In an embodiment, the gas inlet port is coupled to a gas inlet channel, and the gas outlet port is coupled to a gas outlet channel, the gas inlet channel and the gas outlet channel directed to open at the distal end of the elongate shaft body.

In an embodiment, the secondary cleaning module is a wiper arranged to be in contact with an external surface of the aperture cover for improving visualization.

In an embodiment, the wiper is an elongate band coupled on a first end to the elongate shaft body and coupled on a second end to the elongate shaft body and diametrically opposite to the first end.

In an embodiment, the wiper is coupled to a retractable shaft actuated at the proximal end of the elongate shaft body.

In an embodiment, the coupling to the retractable shaft is hinged to elevate the wiper when the retractable shaft is actuated in a first direction and to deploy wiper on the surface of the aperture cover when the retractable shaft is actuated in the opposite direction.

In an embodiment, the wiper is arranged to rotate about the aperture cover.

In an embodiment, the retractable shaft is arranged to rotate about a rotating shaft coupled to the aperture cover.

In an embodiment, the wiper is further hinged to a rotating cylinder for rotating the wiper about the aperture cover.

In an embodiment, the wiper is coupled to a pivoting element provided at a point on the circumference of the elongate shaft body and actuated to rotate the wiper in a sweeping motion across the aperture cover.

In an embodiment, the endoscope further includes a second wiper coupled to a second pivoting element provided at a diametrically opposing point on the circumference of the elongate shaft body as the first pivoting element.

In an embodiment, the second wiper is arranged to rotate in a sweeping motion across the aperture cover in succession to the sweeping motion rotation of the first wiper.

In an embodiment, the second wiper is arranged to rotate in a sweeping motion across the aperture cover in a direction opposing the sweeping motion rotation of the first wiper and in succession to the sweeping motion rotation of the first wiper.

In an embodiment, the wiper is a flexible elongate band coupled at a first end to the elongate shaft body and coupled at a second end to a carrier, the carrier received in a guide track on the circumference of the elongate shaft body.

In an embodiment, the carrier is arranged to traverse from a first position on the guide track where the wiper is deployed across the aperture cover to a second position on the guide track where the wiper is displaced to the circumference of the aperture cover.

In an embodiment, at the first position on the guide track, the carrier is diametrically opposed to the coupling of the first end of the flexible elongate band to the elongate shaft body.

In an embodiment, the flexible elongate band is a resilient band.

In an embodiment, the coupling of the first end of the flexible elongate band to the elongate shaft body is a retractable coupling for maintaining a tension on the flexible elongate band.

In an embodiment, the wiper is a flexible elongate band coupled at a first end and a second end on the elongate shaft body at diametrically opposing points.

In an embodiment, each of the first end and the second end of the flexible elongate band is coupled to an axle, each axle rotatable about an axis parallel to the length of the elongate shaft body.

In an embodiment, the flexible elongate band is arranged for motion only in a plane parallel to the external surface of the aperture cover.

In an embodiment, the axle coupled to the first end of the flexible elongate band is driven to rotate such that the flexible elongate band is actuated from side to side of the elongate shaft bisected by the diametrically opposing coupling points.

In an embodiment, the flexible elongate band is a resilient band.

In an embodiment, the elongate shaft body further includes a drainage channel to allow removal of debris accumulated by the wiper.

In an embodiment, the endoscope further includes a spooling actuation system coupled to the wiper.

In an embodiment, the wiper is elongated and is actuable with the spooling actuation system such that a clean portion of the wiper can replace a soiled portion for cleaning the aperture cover.

In an embodiment, the wiper is coupled at a first point on the circumference of the elongate shaft body and coupled at a second point to a shuttle, the shuttle arranged to traverse about the circumference of the elongate shaft body.

In an embodiment, the endoscope further includes a retention module for receiving a portion of the wiper, the retention module for causing the wiper to contact the external surface of the aperture cover.

In an embodiment, the wiper is any one of a floss, a thread, or a bundle of threads. In an embodiment, the wiper is a floss. In an embodiment, the wiper is a thread. In an embodiment, the wiper is a bundle of threads.

In an embodiment, the wiper is a strip and includes a plurality of regularly spaced holes in the strip.

In an embodiment, the strip is reinforced with a layer of tape on a side of the strip which faces away from the aperture cover.

In an embodiment, an optical lens is provided for mounting over the optical aperture.

In an embodiment, the optical lens is a dome-shaped lens.

In an embodiment, optical lens is the aperture cover.

In an embodiment, the endoscope further includes a pair of arms arranged to retain the wiper over the pair of arms.

In an embodiment, the pair of arms is provided on opposing sides of the optical scope, the pair of arms arranged to be retained within the elongate shaft body.

In an embodiment, the elongate shaft body is arranged to be retractable proximally.

In an embodiment, the pair of arms is arranged to flare open and lower the wiper onto the aperture cover for cleaning.

In an embodiment, the elongate shaft body further includes a set of guides on an inner surface of the elongate shaft body.

In an embodiment, the set of guides is provided by assembling a guide supplement onto the inner surface of the elongate shaft body.

In an embodiment, the set of guides provide an additional point of inflection of the wiper in transiting the wiper from a spooling channel parallel to the elongate shaft body to being contact with the aperture cover.

In an embodiment, the wiper is a clear flexible material for protecting the optical aperture, and is spooled to replace a soiled portion.

The above apparatus, method and/or system as described and illustrated in the corresponding figures, is not intended to limit an or any apparatus, method or system as according to an embodiment, and the scope of the present disclosure. The description further includes, either explicitly or implicitly, various features and advantages of the method or system according to the present disclosure, which can be encompassed within an apparatus, method or system according to the disclosure.

While embodiments of the disclosure have been particularly shown and described with reference to specific embodiments, it should be understood by those skilled in the art that various changes in form and detail may be made therein without departing from the spirit and scope of the disclosure as defined by the appended claims. The scope of the disclosure is thus indicated by the appended claims and all changes which come within the meaning and range of equivalency of the claims are therefore intended to be embraced.

1. An endoscope comprising:
an elongate shaft body;
an optical scope provided within the elongate shaft body, the optical scope comprising an optical aperture provided at the distal end of the optical scope, corresponding to a distal end of the elongate shaft body;
an aperture cover provided at the distal end of the elongate shaft body for shielding the optical aperture from debris external to the elongate shaft body; and
an actuation system provided at a proximal end of the elongate shaft body;
wherein the actuation system drives the aperture cover to rotate.

2. The endoscope of claim 1, wherein the actuation system comprises a motor at a proximal end of the elongate shaft body.

3. The endoscope of claim 1, wherein the motor drives a rotating shaft coupled to the circumference of the aperture cover.

4. The endoscope of claim 1, wherein the actuation system is a fluid flow actuation system.

5. The endoscope of claim 1, wherein the aperture cover is circumferentially coupled to a turbine ring comprising a plurality of blades.

6. The endoscope of claim 1, further comprising any one of a helical gear or a worm gear for angulated operation of the aperture cover.

7. The endoscope of claim 1, wherein the aperture cover is configured to operate at a rotation speed which exceeds the flicker fusion threshold.

8. The endoscope of claim 1, further comprising a secondary cleaning module coupled to the distal end of the elongate shaft body, for cooperation with the aperture cover for improving visualization.

9. The endoscope of claim 8, wherein the secondary cleaning module is a fluid flow actuator arranged to provide a fluid flow over an external surface of the aperture cover.

10. The endoscope of claim 9, wherein the fluid flow actuator is coupled to a fluid port on a distal tip on the elongate shaft body.

11. The endoscope of claim 10, wherein the fluid port is directed to displace debris from in front of the external surface of the aperture cover with a fluid flow.

12. The endoscope of claim 8, wherein the secondary cleaning module is a fluid flow circulator arranged to provide a fluid flow circulation over an external surface of the aperture cover.

13. The endoscope of claim 8, wherein the secondary cleaning module is a wiper arranged to be in contact with an external surface of the aperture cover for improving visualization.

14. The endoscope of claim 13, wherein the wiper is an elongate band coupled on a first end to the elongate shaft body and coupled on a second end to the elongate shaft body and diametrically opposite to the first end.

15. The endoscope of claim 13, wherein the wiper is coupled to a pivoting element provided at a point on the circumference of the elongate shaft body and actuated to rotate the wiper in a sweeping motion across the aperture cover.

16. The endoscope of claim 13, wherein the wiper is a flexible elongate band coupled at a first end to the elongate shaft body and coupled at a second end to a carrier, the carrier received in a guide track on the circumference of the elongate shaft body.

17. The endoscope of claim 16, wherein the coupling of the first end of the flexible elongate band to the elongate shaft body is a retractable coupling for maintaining a tension on the flexible elongate band.

18. The endoscope of claim 13, further comprising a spooling actuation system coupled to the wiper.

19. The endoscope of claim 18, wherein the wiper is a strip and includes a plurality of regularly spaced holes in the strip.

20. The endoscope of claim 1, wherein an optical lens is provided for mounting over the optical aperture and wherein the optical lens is a dome-shaped lens.
